(19) United States
(12) Patent Application Publication
(10) Pub. No.: US 2013/0130240 A1
(43) Pub. Date: May 23, 2013

(54) METHODS FOR IDENTIFYING DRUG RESISTANT MYCOBACTERIUM

(71) Applicants: Tobin J. Hellyer, Westminster, MD (US); James G. Nadeau, Ellicott City, MD (US)

(72) Inventors: Tobin J. Hellyer, Westminster, MD (US); James G. Nadeau, Ellicott City, MD (US)

(73) Assignee: BECTON DICKINSON AND COMPANY, FRANKLIN LAKES, NJ (US)

(21) Appl. No.: 13/574,770

(22) PCT Filed: Dec. 17, 2010
(86) PCT No.: PCT/US10/60917

§ 371 Date: Feb. 5, 2013

Related U.S. Application Data
(60) Provisional application No. 61/288,706, filed on Dec. 21, 2009.

(51) Int. Cl. C12Q 1/68

(52) U.S. Cl. CPC C12Q 1/6816
USPC 435/6.11

(57) ABSTRACT

Presented herein are methods for determining the presence of a non-mutated Mycobacterium rpoB gene core region. For example, presented herein are methods that permit the determination of whether a Mycobacterium is rifampin-resistant by determining whether or not the Mycobacterium rpoB gene core region comprises a mutation. In accordance with such methods, multi-drug-resistant strains of Mycobacterium also can be identified.

### INTRODUCTION

Presented herein are methods for determining the presence of a non-mutated Mycobacterium rpoB gene core region. For example, presented herein are methods that permit the determination of whether a Mycobacterium is rifampin-resistant by determining whether or not the Mycobacterium rpoB gene core region comprises a mutation. In accordance with such methods, multi-drug-resistant strains of Mycobacterium also can be identified.

### BACKGROUND

Tuberculosis is a common and often deadly infectious disease caused by mycobacteria. In humans, Mycobacterium tuberculosis is the primary causative agent of tuberculosis, although other members of the M. tuberculosis complex, e.g., Mycobacterium bovis, Mycobacterium africanum, and Mycobacterium microti also are causes.

Treatment of tuberculosis is often difficult and requires a long course of administration of antimicrobial agents, particularly the first-line antimicrobial agents rifampin and isoniazid, often in combination. However, due to various circumstances (e.g., an improper treatment regimen; low quality medication), drug-resistant Mycobacterium often emerge. Drug-resistant Mycobacterium, particularly multi-drug-resistant Mycobacterium (characterized as being resistant to both rifampin and isoniazid) presents a major public health issue, requiring longer periods of treatment, more extensive and expensive medications for use in the treatment, and administration of second- and third-line drugs that cause/have greater side effects.

The Mycobacterium rpoB gene codes for the beta-subunit of the Mycobacterium RNA polymerase. Rifampin binds to the beta-subunit of the RNA polymerase and prevents the transcription of DNA to RNA by the polymerase, which results in the inhibition of subsequent translation of RNA to proteins. Mutations in a specific region of the Mycobacterium rpoB gene result in the generation of RNA polymerases with subtly different beta-subunit structures which are not readily inhibited by rifampin, thus generating rifampin-resistant strains. Moreover, resistance to rifampin often develops concurrently with resistance to isoniazid (see, e.g., Riska et al., 2000, Int. J. Tuberc Lung Dis., 4(2):S4-S10), thus making the identification of mutated Mycobacterium rpoB genes a possible mechanism for identifying multi-drug-resistant Mycobacterium.

Presently, methods for identifying Mycobacterium strains possessing mutations in the rpoB gene, and thus Mycobacterium strains that are rifampin-resistant, require long periods of time and/or sophisticated and expensive machinery. As such, there remains a need in the art for rapid and simplified methods for identifying mutations in the rpoB gene of Mycobacterium.

### SUMMARY

In one aspect, presented herein are methods for the rapid detection of Mycobacterium strains possessing mutations in the rpoB gene core region using detectably-labeled probes. Methods presented herein reduce the number of detectable labels needed to determine the presence of mutations in the Mycobacterium rpoB gene core region, and thus, decrease the complexity of assay systems to detect Mycobacterium strains possessing mutations in the rpoB gene core region. Since mutations within the rpoB gene core region indicate resistance to the antimicrobial agent rifampin, detection of Mycobacterium strains possessing mutations in the rpoB gene core region can provide information for the selection of an appropriate therapeutic regimen for patients, e.g., patients with tuberculosis. In addition to detecting the presence of mutations in the rpoB gene core region, the methods presented herein can be used to detect the presence of particular species or strains of Mycobacterium, as well as the presence of mutations in Mycobacterium genes other than the rpoB gene that may be associated with drug-resistance.

In one embodiment, presented herein is a method for determining the presence of a non-mutated Mycobacterium rpoB gene core region, comprising:
(a) contacting a nucleic acid sample with a composition comprising a set of probes, wherein at least four probes are rpoB probes that are specific for a non-mutated rpoB gene core region, under conditions that allow hybridization of the rpoB probes to the core region, and wherein the first and third rpoB probes are labeled with a fluorescent donor moiety and the second and fourth rpoB probes are labeled with different fluorescent acceptor moieties, and wherein the rpoB probes hybridize to the non-mutated rpoB gene core region so that the fluorescent acceptor moieties can accept the transfer of energy from the fluorescent donor moieties; and
(b) assaying for the presence of fluorescence emission that results when both donor moieties transfer energy to both acceptor moieties, wherein the presence of the fluorescence emission that results when both donor moieties transfer energy to both acceptor moieties indicates the presence of a non-mutated rpoB gene core region, and wherein the absence of or reduction in the fluorescence emission that results when both donor moieties transfer energy to both acceptor moieties indicates the absence of a non-mutated rpoB gene core region.

In another embodiment, presented herein is a method for determining the presence of Mycobacterium and for determining the presence of a non-mutated Mycobacterium rpoB gene core region, comprising:
(a) contacting a nucleic acid sample with a composition comprising (i) at least one Mycobacterium detector probe, and (ii) a set of probes, wherein at least four probes are rpoB probes that are specific for a non-mutated rpoB gene core region, under conditions that allow hybridization of the rpoB probes to the core region and hybridization of the detector probe to a complementary Mycobacterium nucleic acid sequence, and wherein the first and third rpoB probes are labeled with a fluorescent donor moiety and the second and fourth rpoB probes are labeled with different fluorescent acceptor moieties, and wherein the rpoB probes hybridize to the non-mutated rpoB gene core region so that the fluorescent acceptor moieties can accept the transfer of energy from the fluorescent donor moieties; and
(b) assaying for the presence of hybridized detector probe and the fluorescence emission that results when both donor moieties transfer energy to both acceptor moieties,
wherein (i) the presence of hybridized detector probe and the presence of the fluorescence emission that results when both donor moieties transfer energy to both acceptor moieties indicates the presence of Mycobacterium and the presence of a non-mutated rpoB gene core region; (ii) the absence of hybridized detector probe and the absence of or reduction in the fluorescence emission that results when both donor moieties transfer energy to both acceptor moieties indicates that Mycobacterium is not present; and (iii) the presence of hybridized detector probe and the absence of or reduction in the fluorescence emission that results when both donor moieties transfer energy to both acceptor moieties indicates the presence of Mycobacterium and the absence of a non-mutated rpoB gene core region.

It should be understood that in each of the methods described herein, each of the rpoB probes hybridizes to a different portion of the rpoB gene core region. For example, when four rpoB probes that hybridize to the rpoB gene core region are used in accordance with one of the methods described herein, each of the four rpoB probes hybridizes to a different portion of the core region. Likewise, when three rpoB probes that hybridize to the rpoB gene core region are used in accordance with one of the methods described herein, each of the three rpoB probes hybridizes to a different portion of the core region. In some embodiments, the rpoB probes that hybridize to the rpoB gene core region hybridize such that the entirety of the rpoB gene core region is hybridized by the rpoB probes when there is no mutation in the rpoB gene core region. For example, if an rpoB gene core region comprises 81 base pairs, the entire 81 base pairs of the rpoB gene core region are hybridized to rpoB probes when there is no mutation in the rpoB gene core region. In other embodiments, the rpoB probes that hybridize to the rpoB gene core region hybridize such that the entirety of the rpoB gene core region is not hybridized by the rpoB probes when there is no mutation in the rpoB gene core region, i.e., the rpoB probes, when hybridized, do not cover the entirety of the rpoB gene core region. For example, if an rpoB gene core region comprises 81 base pairs, the number of base pairs of the rpoB gene core region that are hybridized to rpoB probes when there is no mutation in the rpoB gene core region may be less than 81, e.g., 80 or less, 79 or less, 78 or less, 77 or less, 76 or less, 75 or less, 74 or less, 73 or less, 72 or less, 71 or less, 70 or less, 65 or less, 60 or less, or 50 or less base pairs are hybridized to rpoB probes when there is no mutation in the rpoB gene core region.

In accordance with the methods described herein, the presence of a non-mutated rpoB gene core region indicates that the Mycobacterium is susceptible to the antimicrobial agent rifampin, whereas the absence of a non-mutated rpoB gene core region indicates that the Mycobacterium is susceptible to the antimicrobial agent rifampin. In certain embodiments, determination that the Mycobacterium is resistant to rifampin indicates that the Mycobacterium is multi-drug-resistant Mycobacterium.

In some embodiments of the methods described herein, one or more of the rpoB probes may comprise a self-complementary region, such that in the absence of a nucleic acid sample containing nucleic acid that hybridizes to the probe, the probe forms a hairpin loop. In certain embodiments, the probe comprises a quencher moiety that quenches a fluorescent moiety of the probe (e.g., a fluorescent donor moiety) when the probe forms a hairpin loop. In accordance with such embodiments, the fluorescence emitted from the fluorescent moiety of the probe can only be detected if the probe has hybridized to a complementary nucleic acid sequence, thus reducing non-specific detection of the fluorescence emitted from the fluorescent moiety of the probe.

In some embodiments of the methods described herein, the set of probes further comprises blocking probes which increase the specificity of the method. In certain embodiments, a blocking probe is at least partially complementary to an rpoB probe that hybridizes to the rpoB gene core region, so that in the absence of a nucleic acid sample containing nucleic acid that hybridizes to the rpoB probe, the blocking probe hybridizes to the rpoB probe. In some embodiments, a blocking probe is labeled with a quencher moiety that quenches a fluorescent moiety (e.g., a fluorescent donor moiety) of the rpoB probe that hybridizes to the rpoB gene core region. In other embodiments, a blocking probe is at least partially complementary to a nucleic acid sequence of the non-mutated rpoB gene core region, wherein in the absence of one or more rpoB probes that hybridize to the rpoB gene core region the blocking probe hybridizes to the non-mutated rpoB gene core region, and wherein in the presence of the non-mutated rpoB gene core region, the non-mutated rpoB gene core region hybridizes to rpoB probes and not to the blocking probe.

In certain embodiments, a nucleic acid sample is lysed prior to use in a method described herein. In other embodiments, a method described herein comprises lysis of a nucleic acid sample as part of the method, e.g., a lysing agent is included in step (a) of the method.

In certain embodiments, a nucleic acid sample used in the methods described herein comprises amplified nucleic acids. In a specific embodiment, the amplified nucleic acids comprise nucleic acids that correspond to the Mycobacterium rpoB gene or a region of the Mycobacterium rpoB gene. In some embodiments, the amplified nucleic acids comprise nucleic acids that correspond to a gene other than the rpoB gene or a region of a gene other than the rpoB gene, e.g., the nucleic acid amplified is the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene of Mycobacterium, or a region thereof.

In some embodiments, nucleic acids in a nucleic acid sample are amplified prior to use in a method described herein. In other embodiments, nucleic acids in a nucleic acid sample are amplified as part of a method described herein, e.g., an amplification step is included in step (a) of the method. In specific embodiments, the rpoB gene or a region of the rpoB gene of Mycobacterium is amplified. In some embodiments, a gene other than the rpoB gene or a region of a gene other than the rpoB gene, e.g., the nucleic acid amplified is the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene of Mycobacterium, or a region thereof is amplified.

The methods described herein can further comprise detection of mutations in Mycobacterium genes other than rpoB, wherein said mutations correlate with resistance to antimicrobial agents other than rifampin. For example, probes can be used that hybridize to non-mutated regions of these genes, wherein mutations in the regions that the fluorescently-labeled probes hybridize correlate with resistance to a certain antimicrobial agent. In accordance with such methods, if fluorescence emitted from the probe is detected, then there is no mutation in the gene and the Mycobacterium is not indicated to be resistant to the antimicrobial agent in question, whereas if fluorescence emitted from the probe is not detected, then there is a mutation in the gene and the Mycobacterium is indicated to be resistant to the antimicrobial agent in question. Alternatively, probes can be used that hybridize to mutated regions of Mycobacterium genes, other than rpoB, wherein the mutations correlate to resistance to antimicrobial agents other than rifampin. In accordance with such methods, if fluorescence emitted from the probe is detected, then there is a mutation in the gene and the Mycobacterium is indicated to be resistant to the antimicrobial agent in question, whereas if fluorescence emitted from the probe is not detected, then there is not a mutation in the gene and the Mycobacterium is not indicated to be resistant to the antimicrobial agent in question.

Mutations in Mycobacterium genes that confer resistance to antimicrobial agents are well known in the art, e.g., mutations in the katG, inhA, mabA, ahpC, and oxyR genes confer resistance to isoniazid; mutations in the pncA gene confer resistance to pyrazinamide; mutations in the rrs and rpsL genes confer resistance to streptomycin; mutations in the embA, embB, and embC genes confer resistance to ethambutol; mutation in the inhA gene also confers resistance to ethionamide; and mutations in the gyrA, gyrB, and nor genes confer resistance to ciprofloxacin (see, e.g., Drobniewski and Wilson, 1998, J. Med. Microbiol., 47:189-196).

In another aspect, presented herein are kits for use in determining the presence of a non-mutated Mycobacterium rpoB gene core region in accordance with one or more of the methods described herein.

### TERMINOLOGY

As used herein, the terms “about” and “approximately,” unless otherwise indicated, refer to a value that is no more than 20% above or below the value being modified by the term.

As used herein, the term “source” refers to anything which can provide a nucleic acid sample, e.g., a subject, (e.g., a human or non-human animal).

As used herein, the term “nucleic acid sample” refers to a sample that comprises nucleic acid or is suspected to comprise nucleic acid, e.g., nucleic acid of Mycobacterium. A nucleic acid sample may comprise components other than nucleic acid, e.g., host cells of a subject (e.g., a human or non-human animal), and/or a nucleic acid sample may be purified, isolated or concentrated. Methods for the purification, isolation, and concentration of nucleic acids are well-known in the art. In specific embodiments, nucleic acids within a nucleic acid sample are accessible to probes for hybridization. In a specific embodiment, a nucleic acid sample is lysed. Lysis can be performed using any method known in the art, e.g., heat, enzymes, detergents, buffers, acids, bases, chaotropes, physical shearing in the presence of beads or particles, and/or the application of pressure. In some embodiments, a nucleic acid sample may be subjected to one or more denaturing steps, to denature any double-stranded nucleic acids or nucleic acids that possess internal secondary structure. Methods for denaturation of nucleic acids are well-known in the art. In some embodiments, a nucleic acid sample comprises amplified nucleic acids, e.g., an amplified rpoB gene core region. In other embodiments, a nucleic acid sample is amplified as part of one or more of the methods described herein. Methods of nucleic acid amplification are well-known in the art, e.g., PCR, SDA, TMA, NASBA, rolling circle amplification, helix displacement amplification, or LAMP.

As used herein, the term “Mycobacterium” refers to a strain of Mycobacterium comprising an rpoB gene, wherein the rpoB gene comprises a core region and wherein mutations in the rpoB gene core region correlate with resistance to rifampin. In certain embodiments, Mycobacterium refers to members of the Mycobacterium tuberculosis complex, e.g., Mycobacterium tuberculosis, Mycobacterium africanum, Mycobacterium bovis, Mycobacterium bovis BCG, and Mycobacterium microti. The nucleic acid sequences of the rpoB genes for many Mycobacterium have been assigned GenBank accession numbers, e.g., L27989 (the rpoB gene of Mycobacterium tuberculosis); AY544894 (the rpoB gene of Mycobacterium bovis); AF057453 (the rpoB gene of Mycobacterium bovis BCG); AY544944 (the rpoB gene of Mycobacterium microti); and AY544880 (the rpoB gene of Mycobacterium africanum).

As used herein, the term “Mycobacterium rpoB gene core region” refers to the region of the rpoB gene of Mycobacterium that, when comprising a mutation, correlates with resistance to rifampin. In some embodiments, a Mycobacterium rpoB gene core region corresponds to the Mycobacterium rpoB gene core region of the rpoB gene of Mycobacterium tuberculosis, Mycobacterium africanum, Mycobacterium bovis, Mycobacterium bovis BCG, Mycobacterium microti, or Mycobacterium avium. In a specific embodiment, a Mycobacterium rpoB gene core region corresponds to the Mycobacterium rpoB gene core region of the rpoB gene of Mycobacterium tuberculosis, e.g., the core region comprises 81 base pairs and corresponding to codons 507-533 of the Mycobacterium tuberculosis rpoB gene (see, e.g., Hauck et al., 2009, J. Antimic. Chemother., 64:259-262).

As used herein, the terms “non-mutated Mycobacterium rpoB gene core region” and “non-mutated rpoB gene core region” refer to a Mycobacterium rpoB gene core region that does not comprise a mutation that correlates with resistance to the antimicrobial agent rifampin.

As used herein, the term “growth medium” generally relates to a nutrient source which allows Mycobacterium to grow. Growth medium may provide vitamins, amino acids, trace elements, salts, compounds and any other substances, whether organic or inorganic, necessary for or conducive to maintain life and foster replication/reproduction of the Mycobacterium. Exemplary growth media include Middlebrook medium, Lowenstein-Jensen slants, Ogawa Egg Yolk medium, MGIT tubes (Becton Dickinson), and BACTEC 12B (Becton Dickinson).

As used herein, the term “nucleic acid” refers to deoxyribonucleotides, deoxyribonucleic acids, ribonucleotides, and ribonucleic acids, and polymeric forms thereof, and includes either single- or double-stranded forms. Nucleic acids include naturally occurring nucleic acids, such as deoxyribonucleic acid (“DNA”) and ribonucleic acid (“RNA”) as well as nucleic acid analogs. Nucleic acid analogs include those which contain non-naturally occurring bases, nucleotides that engage in linkages with other nucleotides other than the naturally occurring phosphodiester bond or which contain bases attached through linkages other than phosphodiester bonds. Thus, nucleic acid analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), locked-nucleic acids (LNAs), and the like.

As used herein, the term “rpoB probe” refers to a probe that is specific to a portion of the Mycobacterium rpoB gene.

As used herein, the term “Mycobacterium detector probe” refers to a probe that comprises a detectable label that is complementary to a nucleic acid sequence of Mycobacterium other than a nucleic acid sequence of the rpoB gene. In a specific embodiment, a Mycobacterium detector probe is specific for a nucleic acid sequence of Mycobacterium that is conserved among all or multiple species or strains of Mycobacterium. In another embodiment, a Mycobacterium detector probe is specific for a nucleic acid sequence of a particular strain or species of Mycobacterium.

As used herein, the term “absence of or reduction in fluorescence emission,” refers to the phenomenon observed when the fluorescence emitted by a fluorescent moiety either cannot be detected (i.e., it is absent) or is reduced relative to the fluorescence emitted by the fluorescent moiety that is detectable under a given condition, e.g., when the fluorescence emitted by the fluorescent moiety is unquenched or when the fluorescent moiety is positioned in a manner that allows transfer of fluorescence from the fluorescent moiety to a fluorescent acceptor moiety. For example, when a fluorescent donor moiety and a fluorescent acceptor moiety are not positioned in a manner that allows transfer of fluorescence from the fluorescent donor moiety to the fluorescent acceptor moiety, then an absence of or reduction in fluorescence emission will occur, i.e., FRET does not occur. In another example, the fluorescence emitted by a fluorescent moiety may be absent or reduced when the fluorescence emitted by the fluorescent moiety is quenched by quencher moiety.

As used herein, the term “purified” in the context of nucleic acids refers to a nucleic acid which is separated from other constituents (e.g., proteins, lipids salts, other types of nucleic acid molecule) which are present in the natural source of the nucleic acid or to a probe that has been synthesized based on a given nucleic acid sequence and purified from the excess nucleic acids utilized in the synthesis of the probe. In certain embodiments, a purified nucleic acid is at least 60% pure, at least 65% pure, at least 70% pure, at least 75% pure, at least 80% pure, at least 85% pure, at least 90% pure, at least 99% pure, or at least 99.9% pure as assessed by techniques known to one of skill in the art.

As used herein the term “Fluorescence Resonance Energy Transfer” or “FRET” refers to an energy transfer mechanism occurring between two fluorescent molecules: a fluorescent donor moiety and a fluorescent acceptor moiety or quencher moiety (i.e., a FRET pair) wherein one member of the FRET pair (the fluorescent donor moiety) is excited at its specific fluorescence excitation wavelength and transfers the fluorescent energy to a second molecule (the fluorescent acceptor moiety or quencher moiety) and the donor returns to the electronic ground state.

As used herein, the term “fluorescent moiety” refers to a fluorescent compound that emits fluorescence that can be detected.

As used herein, the term “fluorescent donor moiety” refers to a fluorescent compound that can absorb energy and is capable of transferring the energy to a fluorescent acceptor moiety or quencher moiety.

As used herein, the term “fluorescent acceptor moiety” refers to a fluorescent compound that absorbs energy from a fluorescent donor moiety and re-emits the transferred energy as fluorescence. In FRET, a fluorescent acceptor moiety has an adequate spectral overlay with the fluorescent donor moiety to be capable of accepting the energy emitted by the fluorescent donor moiety when the members of the FRET pair (i.e., the fluorescent donor moiety and the fluorescent acceptor moiety) are positioned at the characteristic distance for FRET.

As used herein, the term “quencher moiety” refers to a molecule or a part of a compound that is capable of reducing the emission from a fluorescent moiety (e.g., a fluorescent donor moiety). Such reduction includes reducing the light after the time when a photon is normally emitted from a fluorescent moiety. In FRET, a quencher has an adequate spectral overlay with the fluorescent moiety to be capable of accepting the energy emitted by the fluorescent moiety when the members of the FRET pair (i.e., the fluorescent moiety and the quencher moiety) are positioned at the characteristic distance for FRET. Other non-FRET mechanisms of quenching also exist that do not rely upon spectral overlap between the donor and acceptor moieties (see, e.g., Tyagi et al., 1998, Nature Biotechnol. 16:49-53).

Examples of fluorescent donor moieties, fluorescent acceptor moieties, and quencher moieties may include, but are not limited to, coumarins and related dyes, xanthene dyes (e.g., fluoresceins, rhodols and rhodamines), resorufins, cyanine dyes, bimanes, acridines, isoindoles, dansyl dyes, aminophthalic hydrazides (e.g., luminol and isoluminol derivatives), aminophthalimides, aminonaphthalimides, aminobenzofurans, aminoquinolines, dicyanohydroquinones, fluorescent europium, terbium complexes and related compounds. Specific examples of quencher moieties may include, but are not limited to DABCYL and Black Hole Quenchers I-III. Specific examples of fluorescent donor moieties, fluorescent acceptor moieties, and quencher moieties may include, but are not limited to, FITC, ROX, GFP, Cy5, Cy5.5, Cy3, Cy3B, GFP, YFP, RFP, CFP, Rhodamine Red, Texas Red, Bodipy, IDR700, LightCycler 610, LightCycler 640, LightCycler 670, LightCycler 705, and TAMRA.

As used herein, the terms “quenching” and “quenched” refer to partial or full absorption of energy by a fluorescent acceptor moiety or a quencher moiety. The quenching phenomena may occur between two fluorescent components that are the same or substantially the same (e.g., a single cyanine dye) or two fluorescent components that are different (e.g., a cyanine dye and squarine dye), or between a fluorescent component and a non-fluorescent acceptor (e.g., a fluorescein dye and a DABCYL moiety).

As used herein, the term “pre-determined reference range” refers to a reference range for the readout of a particular assay.

As used herein, the term “detectable label” refers to any label that provides, directly or indirectly, a detectable signal. In some embodiments, a detectable label comprises a biological or chemical molecule, for example, enzymes, radiolabeled molecules, fluorescent molecules, fluorophores (e.g., FITC, ROX, GFP, Cy5, Cy5.5, Cy3, Cy3B, GFP, YFP, RFP, CFP, Rhodamine Red, Texas Red, Bodipy, IDR700, LightCycler 610, LightCycler 640, LightCycler 670, LightCycler 705, and TAMRA), particles, chemiluminesors, enzyme substrates or cofactors, enzyme inhibitors, or magnetic particles. In other embodiments, a detectable label comprises a physical property, e.g., size, shape, electrophoretic mobility, hydrophobicity, hydrophilicity, solubility, and/or chromatographic behavior.

As used herein, the terms “hybridize,” “hybridizes,” and “hybridization” refer to the binding of two or more nucleic acid sequences that are at least 60% (preferably, 70%, 75%, 80%, 85%, 90%, 95%, 98%, 99% or 99.5%) complementary to each other. Nucleic acid hybridization techniques and conditions are known to the skilled artisan and have been described, e.g., in Sambrook et al. Molecular Cloning A Laboratory Manual, 2nd Ed. Cold Spring Lab. Press, December 1989; U.S. Pat. Nos. 4,563,419 and 4,851,330, and in Dunn, et al., Cell 12, pp. 23-26 (1978) among many other publications. Various modifications to the hybridization reactions are known in the art including in-solution hybridization or hybridization to probes on a solid support in one or more reaction steps.

As used herein, the terms “subject” and “patient” are used interchangeably to refer to an animal subject. In one embodiment, the subject is a mammal. In another embodiment, the subject is a non-human animal. In another embodiment, the subject is a human.

### BRIEF DESCRIPTION OF THE DRAWINGS

FIG. 1: Exemplary diagram of how rpoB probes may hybridize to the Mycobacterium rpoB gene core region. P1D: probe 1 with comprising a donor moiety; P2A: probe 2 comprising an acceptor moiety; P3D: probe 3 comprising a donor moiety; P4A: probe 4 comprising an acceptor moiety. 1A: The effect of a mutation in the rpoB gene core region bound by a probe comprising a donor moiety. 1B: The effect of a mutation in the rpoB gene core region bound by a probe comprising an acceptor moiety. 1C: The effect of a mutation in the rpoB gene core region bound by a probe comprising a donor moiety. 1D: The effect of a mutation in the rpoB gene core region bound by a probe comprising an acceptor moiety. FIG. 2 demonstrates how an assay such as that depicted in FIG. 1 can be interpreted.

FIG. 2: Exemplary model of how an assay described herein could be interpreted to determine whether or not a Mycobacterium rpoB gene core region comprises a mutation and is therefore resistant to an antimicrobial agent, e.g., rifampin.

### DETAILED DESCRIPTION

Methods for Determining the Presence of a Non-Mutated Mycobacterium rpoB Gene Core Region

In one aspect, presented herein are methods for the rapid detection of Mycobacterium strains possessing mutations in the rpoB gene core region using detectably-labeled probes. Methods presented herein reduce the number of detectable labels that need to be detected to determine the presence of mutations in the Mycobacterium rpoB gene core region, and thus, reduce the complexity of assays designed for the detection of Mycobacterium strains possessing mutations in the rpoB gene core region. Since mutations within the rpoB gene core region indicate resistance to the antimicrobial agent rifampin, detection of Mycobacterium strains possessing mutations in the rpoB gene core region can provide information for the selection of an appropriate therapeutic regimen for patients with tuberculosis. In addition to detecting the presence of mutations in the rpoB gene core region, the methods presented herein can be used to detect the presence of particular species or strains of Mycobacterium, as well as the presence of mutations in Mycobacterium genes other than the rpoB gene that may be associated with drug-resistance.

In some embodiments, the methods presented herein allow for the use of probe sets that hybridize to the entire Mycobacterium rpoB gene core region, yet only require detection of two different detectable signals, e.g., through the use of FRET. As such, these methods allow for the determination of whether a Mycobacterium rpoB gene core region comprises a mutation without the need for detection of more than two detectable labels.

In certain embodiments, the methods presented herein provide for increased specificity when detecting mutations in the Mycobacterium rpoB gene core region through the use of blocking probes. In accordance with such methods, the use of blocking probes ensures that the rpoB probes hybridize to the Mycobacterium rpoB gene core region only when there are no mutations in the Mycobacterium rpoB gene core region.

In one embodiment, presented herein is a method for determining the presence of a non-mutated Mycobacterium rpoB gene core region, comprising:
(a) contacting a nucleic acid sample with a composition comprising a set of probes, wherein at least five probes are rpoB probes that are specific for a non-mutated rpoB gene core region, under conditions that allow hybridization of the rpoB probes to the core region, and wherein (i) the first and third rpoB probes are labeled with a fluorescent donor moiety, (ii) the second and fourth rpoB probes are labeled with different fluorescent acceptor moieties, and (iii) the fifth probe is labeled with a fluorescent moiety; and wherein the rpoB probes hybridize to the non-mutated rpoB gene core region so that the fluorescent acceptor moieties can accept the transfer of energy from the fluorescent donor moieties; and
(b) assaying for (i) the presence of fluorescence emission that results when both donor moieties transfer energy to both acceptor moieties and (ii) fluorescence from the fifth rpoB probe,
wherein the presence of the fluorescence emission that results when both donor moieties transfer energy to both acceptor moieties and the presence of fluorescence from fifth rpoB probe indicates the presence of a non-mutated rpoB gene core region, and wherein the absence of or reduction in the fluorescence emission that results when both donor moieties transfer energy to both acceptor moieties or the absence of fluorescence from the fifth rpoB probe indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, third, and/or fourth rpoB probes further comprise a quencher moiety.

In one embodiment, presented herein is a method for determining the presence of a non-mutated Mycobacterium rpoB gene core region, comprising:
(a) contacting a nucleic acid sample with a composition comprising a set of probes, wherein at least five probes are rpoB probes that are specific for a non-mutated rpoB gene core region, under conditions that allow hybridization of the rpoB probes to the core region, and wherein (i) the first and third rpoB probes are labeled with a fluorescent donor moiety, (ii) the second and fourth rpoB probes are labeled with different fluorescent acceptor moieties, and (iii) the fifth probe is labeled with a fluorescent moiety and a quencher moiety, wherein, in the absence of the non-mutated rpoB gene core region to which the fifth rpoB probe is specific, the fifth rpoB probe forms a hairpin loop such that fluorescence from the fluorescent moiety is quenched; and wherein the rpoB probes hybridize to the non-mutated rpoB gene core region so that the fluorescent acceptor moieties can accept the transfer of energy from the fluorescent donor moieties; and
(b) assaying for (i) the presence of fluorescence emission that results when both donor moieties transfer energy to both acceptor moieties and (ii) fluorescence from the hybridized rpoB probe that is labeled with a fluorescent moiety,
wherein the presence of the fluorescence emission that results when both donor moieties transfer energy to both acceptor moieties and the presence of fluorescence from the fluorescent moiety indicates the presence of a non-mutated rpoB gene core region, and wherein the absence of or reduction in the fluorescence emission that results when both donor moieties transfer energy to both acceptor moieties or the absence of fluorescence from the fluorescent moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, third, and/or fourth rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least four probes are rpoB probes that are specific for a non-mutated rpoB gene core region, under conditions that allow hybridization of the rpoB probes to the core region, and wherein the first and third rpoB probes are labeled with a fluorescent donor moiety and the second and fourth rpoB probes are labeled with different fluorescent acceptor moieties, and wherein the rpoB probes hybridize to the non-mutated rpoB gene core region so that the fluorescent acceptor moieties can accept the transfer of energy from the fluorescent donor moieties; and
(b) assaying for the presence of fluorescence emission that results when both donor moieties transfer energy to both acceptor moieties,
wherein the presence of the fluorescence emission that results when both donor moieties transfer energy to both acceptor moieties indicates the presence of a non-mutated rpoB gene core region, and wherein the absence of or reduction in the fluorescence emission that results when both donor moieties transfer energy to both acceptor moieties indicates the absence of a non-mutated rpoB gene core region.

In one aspect, the first, second, third, and/or fourth rpoB probes further comprise a quencher moiety.

In another embodiment, presented herein is a method for determining the presence of Mycobacterium and for determining the presence of a non-mutated Mycobacterium rpoB gene core region, comprising:
(a) contacting a nucleic acid sample with a composition comprising (i) a Mycobacterium detector probe, and (ii) a set of probes, wherein at least four probes are rpoB probes that are specific for a non-mutated rpoB gene core region, under conditions that allow hybridization of the rpoB probes to the core region and hybridization of the detector probe to a complementary Mycobacterium nucleic acid sequence, and wherein the first and third rpoB probes are labeled with a fluorescent donor moiety and the second and fourth rpoB probes are labeled with different fluorescent acceptor moieties, and wherein the rpoB probes hybridize to the non-mutated rpoB gene core region so that the fluorescent acceptor moieties can accept the transfer of energy from the fluorescent donor moieties; and
(b) assaying for the presence of hybridized detector probe and the fluorescence emission that results when both donor moieties transfer energy to both acceptor moieties,
wherein (i) the presence of hybridized detector probe and the presence of the fluorescence emission that results when both donor moieties transfer energy to both acceptor moieties indicates the presence Mycobacterium and the presence of a non-mutated rpoB gene core region; (ii) the absence of hybridized detector probe and the absence of or reduction in the fluorescence emission that results when both donor moieties transfer energy to both acceptor moieties indicates that Mycobacterium is not present; and (iii) the presence of hybridized detector probe and the absence of or reduction in the fluorescence emission that results when both donor moieties transfer energy to both acceptor moieties indicates the presence Mycobacterium and the absence of a non-mutated rpoB gene core region.

In another aspect, the first, second, third, and/or fourth rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least four probes are rpoB probes that are specific for a non-mutated rpoB gene core region, under conditions that allow hybridization of the probes to the core region, and wherein the first and second rpoB probes are labeled with different fluorescent moieties, the third rpoB probe is labeled with a fluorescent donor moiety, and the fourth rpoB probe is labeled with a fluorescent acceptor moiety, and wherein the third and fourth rpoB probes hybridize to the non-mutated rpoB gene core region so that the fluorescent acceptor moiety can accept the transfer of energy from the fluorescent donor moiety; and
(b) assaying for the presence of (i) fluorescence emission that results when the donor moiety transfers energy to the acceptor moiety and (ii) fluorescence from the hybridized rpoB probes that are labeled with fluorescent moieties,
wherein the presence of the fluorescence emission that results when the donor moiety transfers energy to the acceptor moiety and the presence of fluorescence from the fluorescent moieties indicates the presence of a non-mutated rpoB gene core region, and wherein the absence of or reduction in the fluorescence emission that results when the donor moiety transfers energy to the acceptor moiety or the absence of fluorescence from one of the fluorescent moieties indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, third, and/or fourth rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least four probes are rpoB probes that are specific for a non-mutated rpoB gene core region, under conditions that allow hybridization of the probes to the core region, and wherein (i) the first and second rpoB probes comprise a fluorescent moiety and a quencher moiety, wherein, in the absence of the non-mutated rpoB gene core region to which the first and second rpoB probes are specific, the first and second rpoB probes form a hairpin loop such that fluorescence from the fluorescent moieties is quenched, (ii) the third rpoB probe is labeled with a fluorescent donor moiety, and (iii) the fourth rpoB probe is labeled with a fluorescent acceptor moiety, and wherein the third and fourth rpoB probes hybridize to the non-mutated rpoB gene core region so that the fluorescent acceptor moiety can accept the transfer of energy from the fluorescent donor moiety; and
(b) assaying for the presence of (i) fluorescence emission that results when the donor moiety transfers energy to the acceptor moiety and (ii) fluorescence from the hybridized rpoB probes that are labeled with fluorescent moieties,
wherein the presence of the fluorescence emission that results when the donor moiety transfers energy to the acceptor moiety and the presence of fluorescence from the fluorescent moieties indicates the presence of a non-mutated rpoB gene core region, and wherein the absence of or reduction in the fluorescence emission that results when the donor moiety transfers energy to the acceptor moiety or the absence of fluorescence from one of the fluorescent moieties indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the third and/or fourth rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least four probes are rpoB probes specific for a non-mutated rpoB gene core region, under conditions that allow hybridization of the probes to the core region, and wherein the rpoB probes comprise different fluorescent moieties and a quencher moiety, and wherein, in the absence of the non-mutated rpoB gene core region to which the rpoB probes are specific, the rpoB probes form a hairpin loop such that fluorescence from the fluorescent moieties is quenched; and
(b) assaying for the presence of fluorescence emission of the fluorescent moieties, wherein the presence of fluorescence emission of the fluorescent moieties indicates the presence of a non-mutated rpoB gene core region, and wherein the absence of or reduction in fluorescence emission of one of the fluorescent moieties indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium.

In another embodiment, presented herein is a method for determining the presence of a non-mutated Mycobacterium rpoB gene core region, comprising:
(a) contacting a nucleic acid sample with a composition comprising a set of probes, wherein at least three probes are rpoB probes that are specific for a non-mutated rpoB gene core region, under conditions that allow hybridization of the probes to the core region, and wherein the first rpoB probe is labeled with a fluorescent moiety, the second rpoB probe is labeled with a fluorescent donor moiety, and the third rpoB probe is labeled with a fluorescent acceptor moiety, and wherein the second and third rpoB probes hybridize to the non-mutated rpoB gene core region so that the fluorescent acceptor moiety can accept the transfer of energy from the fluorescent donor moiety; and
(b) assaying for the presence of (i) fluorescence emission that results when the donor moiety transfers energy to the acceptor moiety and (ii) fluorescence from the hybridized rpoB probe that is labeled with a fluorescent moiety,
wherein the presence of the fluorescence emission that results when the donor moiety transfers energy to the acceptor moiety and the presence of fluorescence from the fluorescent moiety indicates the presence of a non-mutated rpoB gene core region, and wherein the absence of or reduction in the fluorescence emission that results when the donor moiety transfers energy to the acceptor moiety or the absence of fluorescence from the fluorescent moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, and/or third rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least three probes are rpoB probes that are specific for a non-mutated rpoB gene core region, under conditions that allow hybridization of the probes to the core region, and wherein (i) the first rpoB probe comprises a fluorescent moiety and a quencher moiety, wherein, in the absence of the non-mutated rpoB gene core region to which the first rpoB probe is specific, the rpoB probe forms a hairpin loop such that fluorescence from the fluorescent moiety is quenched, (ii) the second rpoB probe is labeled with a fluorescent donor moiety, and (iii) the third rpoB probe is labeled with a fluorescent acceptor moiety, and wherein the second and third rpoB probes hybridize to the non-mutated rpoB gene core region so that the fluorescent acceptor moiety can accept the transfer of energy from the fluorescent donor moiety; and
(b) assaying for the presence of (i) fluorescence emission that results when the donor moiety transfers energy to the acceptor moiety and (ii) fluorescence from the first rpoB probe,
wherein the presence of the fluorescence emission that results when the donor moiety transfers energy to the acceptor moiety and the presence of fluorescence from the first rpoB probe indicates the presence of a non-mutated rpoB gene core region, and wherein the absence of or reduction in the fluorescence emission that results when the donor moiety transfers energy to the acceptor moiety or the absence of fluorescence from the first rpoB probe indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the second and/or third rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least three probes are rpoB probes specific for a non-mutated rpoB gene core region, under conditions that allow hybridization of the probes to the core region, and wherein the rpoB probes comprise different fluorescent moieties and a quencher moiety, and wherein, in the absence of the non-mutated rpoB gene core region to which the rpoB probes are specific, the rpoB probes form a hairpin loop such that fluorescence from the fluorescent moieties is quenched; and
(b) assaying for the presence of fluorescence emission of the fluorescent moieties, wherein the presence of fluorescence emission of the fluorescent moieties indicates the presence of a non-mutated rpoB gene core region, and wherein the absence of or reduction in fluorescence emission of one of the fluorescent moieties indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium.

In another embodiment, presented herein is a method for determining the presence of a non-mutated Mycobacterium rpoB gene core region, comprising:
(a) contacting a nucleic acid sample with a composition comprising a set of probes, wherein at least two probes are rpoB probes specific for a non-mutated rpoB gene core region, under conditions that allow hybridization of the probes to the core region, and wherein the rpoB probes comprise different fluorescent moieties and a quencher moiety, and wherein, in the absence of the non-mutated rpoB gene core region to which the rpoB probes are specific, the rpoB probes form a hairpin loop such that fluorescence from the fluorescent moieties is quenched; and
(b) assaying for the presence of fluorescence emission of the fluorescent moieties,
wherein the presence of fluorescence emission of the fluorescent moieties indicates the presence of a non-mutated rpoB gene core region, and wherein the absence of or reduction in fluorescence emission of one of the fluorescent moieties indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium.

In another embodiment, presented herein is a method for determining the presence of a non-mutated Mycobacterium rpoB gene core region, comprising:
(a) contacting a nucleic acid sample with a composition comprising a set of probes, wherein at least two probes are rpoB probes that are specific for a non-mutated rpoB gene core region, under conditions that allow hybridization of the probes to the core region, and wherein the first rpoB probe is labeled with a fluorescent donor moiety and the second rpoB probe is labeled with a fluorescent acceptor moiety, and wherein the rpoB probes hybridize to the non-mutated rpoB gene core region so that the fluorescent acceptor moiety accepts the transfer of energy from the fluorescent donor moiety; and
(b) assaying for the presence of fluorescence emission that results when the donor moiety transfers energy to the acceptor moiety,
wherein the presence of the fluorescence emission that results when the donor moiety transfers energy to the acceptor moiety indicates the presence of a non-mutated rpoB gene core region, and wherein the absence of or reduction in the fluorescence emission that results when the donor moiety transfers energy to the acceptor moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first and/or second rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein at least one probe is an rpoB probe specific for a non-mutated rpoB gene core region, under conditions that allow hybridization of the probe to the core region, and wherein the rpoB probe comprises a fluorescent moiety and a quencher moiety, and wherein, in the absence of the non-mutated rpoB gene core region, the rpoB probe forms a hairpin loop such that fluorescence from the fluorescent moiety is quenched; and
(b) assaying for the presence of fluorescence emission of the donor moiety,
wherein the presence of fluorescence emission of the fluorescent moiety indicates the presence of a non-mutated rpoB gene core region, and wherein the absence of or reduction in fluorescence emission of the fluorescent moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium.

In certain embodiments, the methods provided herein encompass use of a probe set wherein some of the probes in the probe set are complementary to a region of the rpoB gene outside the Mycobacterium rpoB gene core region and wherein other probes in the probe set are specific for a non-mutated rpoB gene core region. For example, in one embodiment, presented herein is a method for determining the presence of a non-mutated Mycobacterium rpoB gene core region, comprising:
(a) contacting a nucleic acid sample with a composition comprising a set of probes, wherein a first probe is an rpoB probe that is specific for a non-mutated rpoB gene core region and a second probe is complementary to a region of the rpoB gene outside of the core region, under conditions that allow hybridization of the probes to rpoB gene, and wherein the first probe is labeled with a fluorescent donor moiety and the second probe is labeled with a fluorescent acceptor moiety, and wherein the first and second probes hybridize to the rpoB gene so that the fluorescent acceptor moiety can accept the transfer of energy from the fluorescent donor moiety; and
(b) assaying for the presence of fluorescence emission that results when the donor moiety transfers energy to the acceptor moiety,
wherein the presence of the fluorescence emission that results when the donor moiety transfers energy to the acceptor moiety, and wherein the absence of or reduction in the fluorescence emission that results when the donor moiety transfers energy to the acceptor moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first and/or second rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein a first probe is complementary to a region of the rpoB gene outside the core region and second and third probes are rpoB probes that are specific for a non-mutated rpoB gene core region, under conditions that allow hybridization of the probes to the core region, and wherein the first probe is labeled with a fluorescent donor moiety, the second probe is labeled with a fluorescent acceptor moiety, and the third probe is labeled with a fluorescent moiety, and wherein the first and second probes hybridize to the rpoB gene so that the fluorescent acceptor moiety can accept the transfer of energy from the fluorescent donor moiety; and
(b) assaying for the presence of (i) fluorescence emission that results when the donor moiety transfers energy to the acceptor moiety and (ii) fluorescence from the hybridized third probe that is labeled with a fluorescent moiety,
wherein the presence of the fluorescence emission that results when the donor moiety transfers energy to the acceptor moiety and the presence of fluorescence from the fluorescent moiety indicates the presence of a non-mutated rpoB gene core region, and wherein the absence of or reduction in the fluorescence emission that results when the donor moiety transfers energy to the acceptor moiety or the absence of fluorescence from the fluorescent moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, and/or third rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein a first probe is complementary to a region of the rpoB gene outside the core region and second, third, and fourth probes are rpoB probes that are specific for a non-mutated rpoB gene core region, under conditions that allow hybridization of the probes to the core region, and wherein the first and third probes are labeled with a fluorescent donor moiety and the second and fourth probes are labeled with different fluorescent acceptor moieties, and wherein the probes hybridize to the rpoB gene so that the fluorescent acceptor moieties can accept the transfer of energy from the fluorescent donor moieties; and
(b) assaying for the presence of fluorescence emission that results when both donor moieties transfer energy to both acceptor moieties,
wherein the presence of the fluorescence emission that results when both donor moieties transfer energy to both acceptor moieties indicates the presence of a non-mutated rpoB gene core region, and wherein the absence of or reduction in the fluorescence emission that results when both donor moieties transfer energy to both acceptor moieties indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, third, and/or fourth rpoB probes further comprise a quencher moiety.

In another embodiment, presented herein is a method for determining the presence of a non-mutated Mycobacterium rpoB gene core region, comprising:
(a) contacting a nucleic acid sample with a composition comprising a set of probes, wherein a first probe is complementary to a region of the rpoB gene outside the core region and second, third, fourth, and fifth probes are rpoB probes that are specific for a non-mutated rpoB gene core region, under conditions that allow hybridization of the probes to the core region, and wherein the first and third probes are labeled with a fluorescent donor moiety, the second and fourth probes are labeled with different fluorescent acceptor moieties, and the fifth probe is labeled with a fluorescent moiety, and wherein the probes hybridize to the rpoB gene so that the fluorescent acceptor moieties can accept the transfer of energy from the fluorescent donor moieties; and
(b) assaying for the presence of (i) fluorescence emission that results when both donor moieties transfer energy to both acceptor moieties and (ii) fluorescence from the hybridized fifth probe that is labeled with a fluorescent moiety,
wherein the presence of the fluorescence emission that results when both donor moieties transfer energy to both acceptor moieties and the presence of fluorescence from the fluorescent moiety indicates the presence of a non-mutated rpoB gene core region, and wherein the absence of or reduction in the fluorescence emission that results when both donor moieties transfer energy to both acceptor moieties or the absence of fluorescence from the fluorescent moiety indicates the absence of a non-mutated rpoB gene core region.

In one aspect, the method further comprises contacting the nucleic acid sample with a Mycobacterium detector probe and assaying for the presence of hybridized detector probe, wherein the presence of hybridized detector probe indicates the presence of Mycobacterium, and wherein the absence of hybridized detector probe indicates the absence of Mycobacterium. In another aspect, the first, second, third, fourth, and/or fifth rpoB probes further comprise a quencher moiety.

It should be understood that in each of the methods described herein, each of the rpoB probes hybridizes that hybridizes to the rpoB gene core region hybridizes to a different portion of the rpoB gene core region. For example, when four rpoB probes that hybridize to the rpoB gene core region are used in accordance with one of the methods described herein, the four rpoB probes bind to the Mycobacterium rpoB gene core region such that each probe hybridizes to a different portion of the core region. Likewise, when three rpoB probes that hybridize to the rpoB gene core region are used in accordance with one of the methods described herein, the three rpoB probes bind to the Mycobacterium rpoB gene core region such that each probe hybridizes to a different portion of the core region. In some embodiments, the rpoB probes that hybridize to the rpoB gene core region hybridize such that the entirety of the rpoB gene core region is hybridized by the rpoB probes when there is no mutation in the rpoB gene core region. For example, if an rpoB gene core region comprises 81 base pairs, the entire 81 base pairs of the rpoB gene core region are hybridized to rpoB probes when there is no mutation in the rpoB gene core region. In other embodiments, the rpoB probes that hybridize to the rpoB gene core region hybridize such that the entirety of the rpoB gene core region is not hybridized by the rpoB probes when there is no mutation in the rpoB gene core region, i.e., the rpoB probes, when hybridized, do not cover the entirety of the rpoB gene core region. For example, if an rpoB gene core region comprises 81 base pairs, the number of base pairs of the rpoB gene core region that are hybridized to rpoB probes when there is no mutation in the rpoB gene core region may be less than 81, e.g., 80 or less, 79 or less, 78 or less, 77 or less, 76 or less, 75 or less, 74 or less, 73 or less, 72 or less, 71 or less, 70 or less, 65 or less, 60 or less, or 50 or less base pairs are hybridized to rpoB probes when there is no mutation in the rpoB gene core region.

In some embodiments of the methods described herein, one or more of the rpoB probes or one or more of the probes specific to Mycobacterium genes other than the rpoB gene may comprise a self-complementary region, such that in the absence of a nucleic acid sample containing nucleic acid that hybridizes to the probe, the probe forms a hairpin loop. In certain embodiments, the probe comprises a quencher moiety that quenches a fluorescent moiety of the probe when the probe forms a hairpin loop. In accordance with such embodiments, the fluorescence emitted from the fluorescent moiety of the probe can be discriminated from background upon hybridization to a complementary nucleic acid sequence, thus permitting homogeneous detection of the fluorescence emitted from the fluorescent moiety of the probe, without the need for removal of unhybridized probe.

In some embodiments of the methods described herein, the set of probes further comprises blocking probes which increase the specificity of the method. In certain embodiments, a blocking probe is partially complementary to an rpoB probe that hybridizes to the rpoB gene core region, so that in the absence of a nucleic acid sample containing nucleic acid that hybridizes to the rpoB probe, the blocking probe hybridizes to the rpoB probe. In some embodiments, a blocking probe is labeled with a quencher moiety that quenches a fluorescent moiety (e.g., a fluorescent donor moiety) of the rpoB probe that hybridizes to the rpoB gene core region to which the blocking probe binds. In other embodiments, a blocking probe is partially complementary to a nucleic acid sequence of the non-mutated rpoB gene core region, wherein in the absence of one or more rpoB probes that hybridize to the rpoB gene core region the blocking probe hybridizes to the non-mutated rpoB gene core region, and wherein in the presence of the non-mutated rpoB gene core region, the non-mutated rpoB gene core region hybridizes to the rpoB probes and not to the blocking probe.

In accordance with the methods described herein, the presence of a non-mutated rpoB gene core region indicates that the Mycobacterium is susceptible to the antimicrobial agent rifampin, whereas the absence of a non-mutated rpoB gene core region indicates that the Mycobacterium is susceptible to the antimicrobial agent rifampin. In certain embodiments, determination that the Mycobacterium is resistant to rifampin indicates that the Mycobacterium is multi-drug-resistant Mycobacterium.

The methods described herein can further comprise detection of mutations in Mycobacterium genes other than rpoB, wherein said mutations correlate to resistance to antimicrobial agents other than rifampin. Mutations in Mycobacterium genes that confer resistance to antimicrobial agents are well known in the art, e.g., mutations in the katG, inhA, mabA, ahpC, kas A, and oxyR genes confer resistance to isoniazid; mutations in the pncA gene confer resistance to pyrazinamide; mutations in the rrs and rpsL genes confer resistance to streptomycin; mutations in the embA, embB, and embC genes confer resistance to ethambutol; resistance in the inhA gene confer resistance to ethionamide; and mutations in the gyrA, gyrB, and nor genes confer resistance to ciprofloxacin (see, e.g., Drobniewski and Wilson, 1998, J. Med. Microbiol., 47:189-196; Riska et al., 2000, Int. J. Tuberc. Lung. Dis., 4(2):S4-S10). Those skilled in the art will understand how the methods provided herein can be modified to include steps for the detection of mutations in any Mycobacterium genes, thus allowing for the determination of whether or not a Mycobacterium is resistant to rifampin and one or more other antimicrobial agents.

In some embodiments, the methods provided herein comprise detection of one or more mutations in the Mycobacterium katG, inhA, mabA, ahpC, kas A, and/or oxyR gene, wherein said mutations are known to or are determined to correlate with resistance of the Mycobacterium to the antimicrobial agent isoniazid. In one embodiment, the mutation is detected using one or more fluorescently-labeled probes specific to a region of the gene that is known to confer resistance to isoniazid, such that the probe hybridizes only when there is no mutation present. The fluorescence of the probe(s) then can be assayed for, wherein the presence of fluorescence emission from the probe(s) indicates the presence of a non-mutated region of the Mycobacterium katG, inhA, mabA, ahpC, kas A, and/or oxyR gene, and wherein the absence of fluorescence from the probe(s) indicates the absence of a non-mutated region of the Mycobacterium katG, inhA, mabA, ahpC, kas A, and/or oxyR gene. In accordance with this embodiment, the presence of fluorescence from the probe(s) indicates that the Mycobacterium is not resistant to the antimicrobial agent isoniazid. In another embodiment, the mutation is detected using one or more fluorescently-labeled probes specific to a region of the gene that is known to confer resistance to isoniazid, such that the probe hybridizes only when there is a mutation present. The fluorescence of the probe(s) then can be assayed for, wherein the presence of fluorescence emission from the probe(s) indicates the absence of a non-mutated region of the Mycobacterium katG, inhA, mabA, ahpC, kas A, and/or oxyR gene, and wherein the absence of fluorescence from the probe(s) indicates the presence of a non-mutated region of the Mycobacterium katG, inhA, mabA, ahpC, kas A, and/or oxyR gene. In accordance with this embodiment, the absence of fluorescence from the probe(s) indicates that the Mycobacterium is not resistant to the antimicrobial agent isoniazid.

In other embodiments, the methods provided herein comprise detection of one or more mutations in the Mycobacterium pncA gene, wherein said mutations are known to or are determined to correlate with resistance of the Mycobacterium to the antimicrobial agent pyrazinamide. In one embodiment, the mutation is detected using one or more fluorescently-labeled probes specific to a region of the gene that is known to confer resistance to pyrazinamide, such that the probe hybridizes only when there is no mutation present. The fluorescence of the probe(s) then can be assayed for, wherein the presence of fluorescence emission from the probe(s) indicates the presence of a non-mutated region of the Mycobacterium pncA gene, and wherein the absence of fluorescence from the probe(s) indicates the absence of a non-mutated region of the Mycobacterium pncA gene. In accordance with this embodiment, the presence of fluorescence from the probe(s) indicates that the Mycobacterium is not resistant to the antimicrobial agent pyrazinamide. In another embodiment, the mutation is detected using one or more fluorescently-labeled probes specific to a region of the gene that is known to confer resistance to pyrazinamide, such that the probe hybridizes only when there is a mutation present. The fluorescence of the probe(s) then can be assayed for, wherein the presence of fluorescence emission from the probe(s) indicates the absence of a non-mutated region of the Mycobacterium pncA gene, and wherein the absence of fluorescence from the probe(s) indicates the presence of a non-mutated region of the Mycobacterium pncA gene. In accordance with this embodiment, the absence of fluorescence from the probe(s) indicates that the Mycobacterium is not resistant to the antimicrobial agent pyrazinamide.

In other embodiments, the methods provided herein comprise detection of one or more mutations in the Mycobacterium rrs and/or rpsL gene, wherein said mutations are known to or are determined to correlate with resistance of the Mycobacterium to the antibiotic streptomycin. In one embodiment, the mutation is detected using one or more fluorescently-labeled probes specific to a region of the gene that is known to confer resistance to streptomycin, such that the probe hybridizes only when there is no mutation present. The fluorescence of the probe(s) then can be assayed for, wherein the presence of fluorescence emission from the probe(s) indicates the presence of a non-mutated region of the Mycobacterium rrs and/or rpsL gene, and wherein the absence of fluorescence from the probe(s) indicates the absence of a non-mutated region of the Mycobacterium rrs and/or rpsL gene. In accordance with this embodiment, the presence of fluorescence from the probe(s) indicates that the Mycobacterium is not resistant to the antibiotic streptomycin. In another embodiment, the mutation is detected using one or more fluorescently-labeled probes specific to a region of the gene that is known to confer resistance to streptomycin, such that the probe hybridizes only when there is a mutation present. The fluorescence of the probe(s) then can be assayed for, wherein the presence of fluorescence emission from the probe(s) indicates the absence of a non-mutated region of the Mycobacterium rrs and/or rpsL gene, and wherein the absence of fluorescence from the probe(s) indicates the presence of a non-mutated region of the Mycobacterium rrs and/or rpsL gene. In accordance with this embodiment, the absence of fluorescence from the probe(s) indicates that the Mycobacterium is not resistant to the antibiotic streptomycin.

In other embodiments, the methods provided herein comprise detection of one or more mutations in the Mycobacterium embA, embB, and/or embC gene, wherein said mutations are known to or are determined to correlate with resistance of the Mycobacterium to the antimicrobial agent ethambutol. In one embodiment, the mutation is detected using one or more fluorescently-labeled probes specific to a region of the gene that is known to confer resistance to ethambutol, such that the probe hybridizes only when there is no mutation present. The fluorescence of the probe(s) then can be assayed for, wherein the presence of fluorescence emission from the probe(s) indicates the presence of a non-mutated region of the Mycobacterium embA, embB, and/or embC gene, and wherein the absence of fluorescence from the probe(s) indicates the absence of a non-mutated region of the Mycobacterium embA, embB, and/or embC gene. In accordance with this embodiment, the presence of fluorescence from the probe(s) indicates that the Mycobacterium is not resistant to the antimicrobial agent ethambutol. In another embodiment, the mutation is detected using one or more fluorescently-labeled probes specific to a region of the gene that is known to confer resistance to ethambutol, such that the probe hybridizes only when there is a mutation present. The fluorescence of the probe(s) then can be assayed for, wherein the presence of fluorescence emission from the probe(s) indicates the absence of a non-mutated region of the Mycobacterium embA, embB, and/or embC gene, and wherein the absence of fluorescence from the probe(s) indicates the presence of a non-mutated region of the Mycobacterium embA, embB, and/or embC gene. In accordance with this embodiment, the absence of fluorescence from the probe(s) indicates that the Mycobacterium is not resistant to the antimicrobial agent ethambutol.

In other embodiments, the methods provided herein comprise detection of one or more mutations in the Mycobacterium inhA gene, wherein said mutations are known to or are determined to correlate with resistance of the Mycobacterium to the antimicrobial agent ethionamide. In one embodiment, the mutation is detected using one or more fluorescently-labeled probes specific to a region of the gene that is known to confer resistance to ethionamide, such that the probe hybridizes only when there is no mutation present. The fluorescence of the probe(s) then can be assayed for, wherein the presence of fluorescence emission from the probe(s) indicates the presence of a non-mutated region of the Mycobacterium inhA gene, and wherein the absence of fluorescence from the probe(s) indicates the absence of a non-mutated region of the Mycobacterium inhA gene. In accordance with this embodiment, the presence of fluorescence from the probe(s) indicates that the Mycobacterium is not resistant to the antimicrobial agent ethionamide. In another embodiment, the mutation is detected using one or more fluorescently-labeled probes specific to a region of the gene that is known to confer resistance to ethionamide, such that the probe hybridizes only when there is a mutation present. The fluorescence of the probe(s) then can be assayed for, wherein the presence of fluorescence emission from the probe(s) indicates the absence of a non-mutated region of the Mycobacterium inhA gene, and wherein the absence of fluorescence from the probe(s) indicates the presence of a non-mutated region of the Mycobacterium inhA gene. In accordance with this embodiment, the absence of fluorescence from the probe(s) indicates that the Mycobacterium is not resistant to the antimicrobial agent ethionamide.

In other embodiments, the methods provided herein comprise detection of one or more mutations in the Mycobacterium gyrA, gyrB, and/or nor gene, wherein said mutations are known to or are determined to correlate with resistance of the Mycobacterium to the antimicrobial agent ciprofloxacin. In one embodiment, the mutation is detected using one or more fluorescently-labeled probes specific to a region of the gene that is known to confer resistance to ciprofloxacin, such that the probe hybridizes only when there is no mutation present. The fluorescence of the probe(s) then can be assayed for, wherein the presence of fluorescence emission from the probe(s) indicates the presence of a non-mutated region of the Mycobacterium gyrA, gyrB, and/or nor gene, and wherein the absence of fluorescence from the probe(s) indicates the absence of a non-mutated region of the Mycobacterium gyrA, gyrB, and/or nor gene. In accordance with this embodiment, the presence of fluorescence from the probe(s) indicates that the Mycobacterium is not resistant to the antimicrobial agent ciprofloxacin. In another embodiment, the mutation is detected using one or more fluorescently-labeled probes specific to a region of the gene that is known to confer resistance to ciprofloxacin, such that the probe hybridizes only when there is a mutation present. The fluorescence of the probe(s) then can be assayed for, wherein the presence of fluorescence emission from the probe(s) indicates the absence of a non-mutated region of the Mycobacterium gyrA, gyrB, and/or nor gene, and wherein the absence of fluorescence from the probe(s) indicates the presence of a non-mutated region of the Mycobacterium gyrA, gyrB, and/or nor gene. In accordance with this embodiment, the absence of fluorescence from the probe(s) indicates that the Mycobacterium is not resistant to the antimicrobial agent ciprofloxacin.

In accordance with the methods described herein that comprise detection of one or more mutations in more than one Mycobacterium gene other than the rpoB gene, it should be understood that the probes specific to the Mycobacterium genes other than the rpoB gene comprise fluorescent labels that are distinct from those that are used to label the one or more rpoB probes used in accordance with the method, i.e., the rpoB probes and the probes specific to the Mycobacterium genes other than the rpoB gene can be detected separately from one another.

In some embodiments, the methods described herein comprise detection of one or more mutations in more than one Mycobacterium gene other than the rpoB gene, wherein the mutations in the different genes correlate with resistance to different antimicrobial agents. For example, a method may comprise detection of one or more mutations in the katG gene, wherein the one or more mutations correlate with resistance to isoniazid; and the method may comprise detection of one or more mutations in the gyrA gene, wherein the one or more mutations correlate with resistance to ciprofloxacin. In accordance with such embodiments, the method allows for the detection of Mycobacterium that is resistant to multiple antimicrobial agents, e.g., rifampin, isoniazid, and ciprofloxacin. In accordance with such embodiments, one or more of the probes specific to Mycobacterium genes other than the rpoB gene may comprise a self-complementary region, such that in the absence of a nucleic acid sample containing nucleic acid that hybridizes to the probe, the probe forms a hairpin loop.

In certain embodiments, the methods described herein comprise one or more wash steps, e.g., the method comprises a wash step before and/or after the hybridization step. In other embodiments, the methods described herein do not comprise a wash step.

In certain embodiments, a nucleic acid sample is lysed prior to use in a method described herein. In other embodiments, a method described herein comprises lysis of a nucleic acid sample as part of the method, e.g., a lysing agent is included in step (a) of the method.

In certain embodiments, a nucleic acid sample used in the methods described herein comprises amplified nucleic acids. In a specific embodiment, the amplified nucleic acids comprise nucleic acids that correspond to the Mycobacterium rpoB gene or a region of the Mycobacterium rpoB gene. In some embodiments, the amplified nucleic acids comprise nucleic acids that correspond to a gene other than the rpoB gene or a region of a gene other than the rpoB gene, e.g., the nucleic acid amplified is the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene of Mycobacterium, or a region thereof. In other embodiments the amplified nucleic acids comprise sequences that are Mycobacterium-specific and which may be used for identification, e.g., IS6110, 16S rRNA, gyrB, dnaJ, or 85B antigen.

In some embodiments, nucleic acids in a nucleic acid sample are amplified prior to use in a method described herein. In other embodiments, nucleic acids in a nucleic acid sample are amplified as part of a method described herein, e.g., an amplification step is included in step (a) of the method. In specific embodiments, the rpoB gene or a region of the rpoB gene of Mycobacterium is amplified. In some embodiments, a gene other than the rpoB gene or a region of a gene other than the rpoB gene, e.g., the nucleic acid amplified is the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene of Mycobacterium, or a region thereof is amplified. In certain embodiments, the methods presented herein comprise lysing of a nucleic acid sample followed by purification of the nucleic acid sample and amplification of the nucleic acid sample before the nucleic acid sample is contacted with one or more of the probes used herein (e.g., rpoB probes and/or probes specific to Mycobacterium genes other than rpoB). For example, a nucleic acid sample may be lysed, purified, and then amplified. Subsequently, the nucleic acid sample may be subjected to one or more of the methods presented herein.

In other embodiments, the methods presented herein comprise lysing of a nucleic acid sample followed by amplification of the nucleic acid sample at the same time that the nucleic acid sample is contacted with one or more of the probes used herein (e.g., rpoB probes and/or probes specific to Mycobacterium genes other than rpoB). For example, a nucleic acid sample may be combined with a lysing agent, amplification agents, and a set of probes and subjected to a method herein, e.g., a method described herein can comprise lysis, amplification, and detection in a single tube reaction.

In other embodiments, the methods presented herein comprise lysing of a nucleic acid sample prior to the method, followed by amplification of the nucleic acid sample at the same time that the nucleic acid sample is contacted with one or more of the probes used herein (e.g., rpoB probes and/or probes specific to Mycobacterium genes other than rpoB). For example, a nucleic acid sample may be lysed and the lysed nucleic acid sample then may be combined with amplification agents, and a set of probes and subjected to a method herein, e.g., a method described herein can comprise amplification and detection in a single tube reaction.

Probes

A probe can refer to any nucleic acid sequence that is designed to specifically hybridize to a target nucleic acid sequence of interest. Typically, but not exclusively, a probe is associated with a detectable label so that the probe (and therefore its nucleic acid target) can be detected, visualized, measured and/or quantified.

The probes used in accordance with the methods described herein can be based on, derived from, or consist of any probes known in the art or can be developed based on any Mycobacterium nucleic acid sequence known or determined, or an analog thereof.

In certain embodiments, the probes used in accordance with the methods described herein may comprise DNA or RNA oligonucleotides and may be chemically synthesized using techniques known in the art. In a specific embodiment, the probes used in accordance with the methods described herein are peptide-nucleic acid (PNA) probes. PNAs are well known by those of skill in the art (see, e.g., Egholm et al., (1993) Nature, 365:566-568). In another specific embodiment, the probes used in accordance with the methods described herein are locked nucleic acid (LNA) probes. PNAs are well known by those of skill in the art (see, e.g., Koshkin et al., (1998) J. Am. Chem. Soc., 120:13252-13253).

Probes of a defined sequence may be produced by techniques known to those of ordinary skill in the art, such as by chemical or biochemical synthesis, and by in vitro or in vivo expression from recombinant nucleic acid molecules, e.g., bacterial or retroviral vectors. The probes encompassed herein exhibit specificity, under hybridization conditions, for their complementary nucleic acid sequences and can be designed to hybridize to deoxyribonucleic acid (DNA) or ribonucleic acid (RNA) e.g., ribosomal RNA (rRNA) or messenger RNA (mRNA).

The probes used in accordance with the methods described herein may hybridize to a nucleic acid sequence of any length so long as hybridization takes place. For example, a probe may hybridize to a nucleic acid sequence that is between 5 to 15 bases in length, 5 to 25 bases in length, 5 to 50 bases in length, 5 to 100 bases in length, 5 to 250 bases in length, 5 to 500 bases in length, 5 to 1000 bases in length, 20 to 25 bases in length, 20 to 50 bases in length, 20 to 100 bases in length, 20 to 250 bases in length, 20 to 500 bases in length, or 20 to 1000 bases in length. In a specific embodiment, the probes used in accordance with the methods described herein hybridize to a nucleic acid sequence that is 15 to 25 bases in length. In another specific embodiment, the probes used in accordance with the methods described herein hybridize to a nucleic acid sequence that is 18 to 22 bases in length. In another specific embodiment, the probes used in accordance with the methods described herein hybridize to a nucleic acid sequence that is about 20 bases in length.

In certain embodiments, a probe used in accordance with the methods described herein is about 5 to 1,000 nucleotides, about 5 to 750 nucleotides, about 5 to 500 nucleotides, about 5 to 250 nucleotides, about 5 to 200 nucleotides, about 5 to 150 nucleotides, about 5 to 100 nucleotides, about 5 to 75 nucleotides, about 10 to 100 nucleotides, about 10 to 75 nucleotides, about 10 to 50 nucleotides, about 10 to 30 nucleotides, about 5 to 20 nucleotides, about 20 to 100 nucleotides, about 20 to 75 nucleotides, or about 30 to 100 nucleotides, in length, or any length in between. In other embodiments, a probe is about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In a specific embodiment, a probe used in accordance with the methods described herein is 15 to 25 bases in length. In another specific embodiment, a probe used in accordance with the methods described herein is 18 to 22 bases in length. In another specific embodiment, a probe used in accordance with the methods described herein is about 20 bases in length. In another specific embodiment, a probe used in accordance with the methods described herein is about 81 nucleotides in length.

In certain embodiments, a probe used in accordance with the methods described herein is at most 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, or 100 nucleotides in length. In other embodiments, a probe is less than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 75, 76, 77, 78, 79, 80, 81, 82, 83, 84, 85, or 90 nucleotides in length.

In certain embodiments, the probes used in accordance with the methods described herein hybridize to their complementary nucleic acid sequences under high stringency, intermediate, or lower stringency hybridization conditions, wherein the choice of hybridization conditions used determines the degree of stringency of hybridization. Optimal hybridization conditions will depend on the length and type (e.g., RNA, or DNA) of probe and nucleic acid to which the probe hybridizes. Those of skill in the art will appreciate that as probes become shorter, it may become necessary to adjust their length to achieve a relatively uniform melting temperature for satisfactory hybridization results. General parameters for stringent hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). In specific embodiments, the probes used in accordance with the methods described herein hybridize to their complementary nucleic acid sequences only under high stringency. Examples of high stringency conditions include: low salt concentration (e.g., 1-250 mM Na+), high temperature relative to the melting temperature of the probe(s) (e.g., from 5° C. below the melting temperature to 5° C. above the melting temperature), high pH (e.g., greater than pH 10), the presence of co-solvents (e.g., 1-20% DMSO or glycerol).

In some embodiments, a probe used in accordance with the methods described herein may include one or more additional nucleic acid sequences that do not hybridize to the complementary nucleic acid sequence of the probe. More than one additional nucleic acid sequence may be included if the first sequence is incorporated into, for example, a self-hybridizing probe (i.e., a probe having distinct base regions capable of hybridizing to each other in the absence of a complementary nucleic acid sequence under the conditions of an assay), such as those used in FRET. In some embodiments, the nucleic acid sequence to which a probe hybridizes comprises additional nucleic acids that the probe does not hybridize to. In other embodiments, the nucleic acid sequence to which a probe hybridizes does not comprise additional nucleic acids that the probe does not hybridize to.

The probes used in accordance with the methods described herein may hybridize to their complementary nucleic acid sequences with varying degrees of specificity. In certain embodiments, the probes hybridize with nucleic acid sequences that are 100% complementary to that of the probe. In other embodiments, the probes hybridize with nucleic acid sequence that are greater than 90% complementary to that of the probe. In other embodiments, the probes hybridize with nucleic acid sequence that are greater than 85% complementary to that of the probe. In other embodiments, the probes hybridize with nucleic acid sequence that are greater than 80% complementary to that of the probe. In other embodiments, the probes hybridize with nucleic acid sequence that are greater than 75% complementary to that of the probe. In other embodiments, the probes hybridize with nucleic acid sequence that are greater than 70% complementary to that of the probe. In other embodiments, the probes hybridize with nucleic acid sequence that are greater than 60% complementary to that of the probe.

The probes used in accordance with the methods described herein can be used at any concentration or amount, so long as the concentration or amount of probe used is sufficient to achieve the desired result of the method. In certain embodiments, the amount of probe used is about 0.001 μg, about 0.01 μg, about 0.1 μg, about 1.0 μg, or more than 1.0 μg. To achieve desired concentrations of probes, the probes can be suspended in a buffer suitable for use in the methods described herein. Exemplary concentrations of probes used herein include 0.0001 μg/ml of probe, 0.001 μg/ml of probe, 0.01 μg/ml of probe, 0.1 μg/ml of probe, 1.0 μg/ml of probe, and 10.0 μg/ml of probe. In certain embodiments, the amount of probe used is about 1.0 nM, 10 nM, 50 nM, 100 nM, 200 nM, 300 nM, 400 nM, 500 nM, 600 nM, 700 nM, 800 nM, 900 nM, 1000 nM, 2000 nM, 3000 nM, 4000 nM, 5000 nM, or greater than 5000 nM. In other embodiments, the amount of probe used is between 1.0 nM and 5000 nM, 10 nM and 5000 nM, 10 nM and 2500 nM, 10 nM and 1000 nM, 50 nM and 5000 nM, 50 nM and 2500 nM, or 50 nM and 1000 nM.

In certain embodiments, the probes used in accordance with the methods described herein comprise a detectable label. A detectable label refers to a moiety, such as a luminescent, fluorescent, or radioactive isotope or group containing the same, and nonisotopic labels, such as enzymes or dyes. In some embodiments, a detectable label comprises a fluorescent donor moiety, a fluorescent acceptor moiety, or a quencher moiety. In a specific embodiment, the probes used in accordance with the methods described herein comprise a fluorescent label. In another specific embodiment, the probes used in accordance with the methods described herein comprise a fluorescent label and a quencher moiety. The probes may be labeled with a detectable label either directly or indirectly. Indirect labeling methods are well known in the art (see, generally, Current Protocols in Immunology, Coligan et al., eds., 1997, John Wiley & Sons, Inc. U.S.A., pp. 8.10.12-8.10.21). Direct methods for linking detectable labels to probes are well known to those of skill in the art. For example, European Patent Publication No. EP 0370 694 A2, entitled, “Diagnostic Kit and Method Using a Solid Phase Capture Means For Detecting Nucleic Acid”, by Burdick and Oakes, publication date May 30, 1990, discloses methods of linking labels to probes. For example, direct labeling can be accomplished by incorporating fluorescent dye-labeled phosphoramidites into a probe during synthesis. Fluorescently labeled nucleoside analogs are available commercially, or can be produced synthetically, by methods known in the art (see, Brumbaugh et al., 1988, Proc. Natl. Acad. Sci. (USA), 85:5610-5614).

In specific embodiments, wherein the probes used in accordance with the methods described herein comprise a fluorescent label, the fluorescent signal that is emitted from the probe in the presence of a complementary nucleic acid sequence is at least about 5%, 10%, 20%, 30%, 40%, 50%, 60%, 70%, 80%, 90%, or 100% higher than the fluorescent signal that is emitted from the probe in the absence of the complementary nucleic acid sequence. In specific embodiments, the fluorescent signal that is emitted from the probe in the presence of the complementary nucleic acid sequence is at least about 1 fold, 1.5 fold, 2 fold, 3 fold, 4 fold, 5 fold, 10 fold, 15 fold, 20 fold, 50 fold, 100 fold, 500 fold, or 1,000 fold higher than the fluorescent signal that is emitted from the probe in the absence of the complementary nucleic acid sequence.

In one embodiment, a probe may comprise one or more fluorescent moieties. In another embodiment, a probe may comprise one or more donor moieties. In another embodiment, a probe may comprise one or more quencher moieties. In another embodiment, a probe may comprise one or more fluorescent acceptor moieties. In one embodiment, a probe may comprise a fluorescent moiety and a quencher moiety positioned in the probe so that the quencher moiety and the fluorescent moiety are brought together in the absence of a complementary nucleic acid sequence. In another embodiment, a probe may comprise a fluorescent donor moiety and a fluorescent acceptor moiety positioned in the probe so that the fluorescent acceptor moiety and fluorescent donor moiety are brought together in the absence of a complementary nucleic acid sequence. For example, the fluorescent donor moiety may be positioned at one terminus of a probe and the quencher moiety or fluorescent acceptor moiety may be positioned at the other terminus of the probe, wherein the probe adopts one conformation or secondary structure, such as a stem-loop or hairpin loop, when not bound or hybridized to a complementary nucleic acid, and adopts a different conformation or secondary structure when bound or hybridized to a complementary nucleic acid. Upon binding between the probe and the complementary nucleic acid, the quencher moiety or fluorescent acceptor moiety and the fluorescent donor moiety separate, resulting in dequenching of the fluorescent signal. In another embodiment, a probe may comprise a fluorescent donor moiety and a fluorescent acceptor moiety positioned in the probe so that when the probe is hybridized to a nucleic acid sequence, the fluorescent acceptor moiety can accept fluorescence from a fluorescent donor moiety of an adjacent probe that is hybridized to the nucleic acid sequence and the fluorescent donor moiety can transfer fluorescence to a fluorescent acceptor moiety of an adjacent probe that is hybridized to the nucleic acid sequence.

In certain embodiments, a probe can be labeled with a single fluorescent moiety, a single fluorescent donor moiety, a single fluorescent acceptor moiety, or a single quencher moiety. In other embodiments, a probe can be labeled with more than one fluorescent moiety, more than one fluorescent donor moiety, more than one fluorescent acceptor moiety, or more than one quencher moiety. In certain embodiments, a probe can be labeled with a single pair of fluorescent moieties and quencher moieties or fluorescent donor moieties and fluorescent acceptor moieties. In other embodiments, a probe can be labeled with different pairs of fluorescent donor moieties and fluorescent acceptor moieties or fluorescent moieties and quencher moieties.

In order to obtain FRET between the fluorescent donor moiety and the fluorescent acceptor moiety or quencher moiety, the two moieties have to be in spatial proximity with each other. Thus, in certain embodiments, a probe comprises both a fluorescent donor moiety and a fluorescent acceptor moiety or quencher moiety such that the fluorescent donor moiety and the fluorescent acceptor moiety or quencher moiety are at most 0.5 nm, at most 1 nm, at most 5 nm, at most 10 nm, at most 15 nm, or at most 20 nm apart from each other. In other embodiments, the distance between a quencher moiety or fluorescent acceptor moiety and the fluorescent donor moiety on a probe is about 10, 15, 20, 30, 40, 50, 60, 70, 80, 90, 100, 110, 105, 110, 115, 120, 130, 140, or 150 A, or any value in between. In other embodiments, a probe comprises both a fluorescent donor moiety and the fluorescent acceptor moiety or quencher moiety such that the fluorescent donor moiety and the fluorescent acceptor moiety or a quencher moiety are at least 2, at least 3, at least 4, at least 5, at least 6, at least 7, at least 8, at least 9, at least 10, at least 15, at least 20, at least 50, at least 100, or at least 200 nucleotides apart from one another. In other embodiments, a probe comprises both a fluorescent donor moiety and the fluorescent acceptor moiety or quencher moiety such that the fluorescent donor moiety and the fluorescent acceptor moiety or a quencher moiety are at least 2 to 5, 2 to 10, 2 to 15, 2 to 20, 3 to 5, 3 to 10, 3 to 15, 3 to 20, 5 to 10, 5 to 20, 5 to 50, 5 to 100, 5 to 200, 10 to 20, 10 to 50, 10 to 100, 10 to 200, 10 to 300, 10 to 400, 10 to 500, 50 to 100, 50 to 250, 50 to 500, 100 to 200, 100 to 300, 100 to 400, or 100 to 500 nucleotides apart from one another.

rpoB Probes

Probes that are specific for the rpoB gene of Mycobacterium are used in accordance with the methods described herein. Probes specific for the rpoB gene of Mycobacterium can be made using any method known in the art or described above. In a specific embodiment, the probes specific for the rpoB gene of Mycobacterium are designed to hybridize to the non-mutated core region of the rpoB gene. In other embodiments, the probes specific for the rpoB gene of Mycobacterium are designed to hybridize to a region of the rpoB gene that does not comprise the core region.

In specific embodiments, the probes specific to the non-mutated Mycobacterium rpoB gene core region are designed so that they only hybridize to their complementary nucleic acid sequence when they are one hundred percent complementary, i.e., the probes will not bind to the nucleic acid sequence of the rpoB gene core region that they are complementary to if the nucleic acid sequence comprises a mutation. In other embodiments, the probes specific to regions of the Mycobacterium rpoB gene that do not comprise the core region may be designed so that they do hybridize to their complementary nucleic acid sequence even if they are not one hundred percent complementary, i.e., the probes may still bind to the nucleic acid sequence of the rpoB gene that does not comprise the core region that they are complementary to even if the nucleic acid sequence comprises a mutation.

In certain embodiments, assay conditions may be selected such that the probes specific to the non-mutated Mycobacterium rpoB gene core region only hybridize to their complementary nucleic acid sequence under specific hybridization conditions (e.g., high-stringency conditions). In other embodiments, assay conditions may be selected such that the probes specific to regions of the Mycobacterium rpoB gene that do not comprise the core region hybridize to their complementary nucleic acid sequence even if they are not fully complementary, i.e., the conditions for hybridization are low- to medium-stringency. Those of skill in the art will understand that low, medium and high stringency conditions are contingent upon multiple factors all of which interact and are also dependent upon the specific designs of the probes in question. For example, for typical probes, high stringency conditions may include temperatures within 5° C. melting temperature of the probe(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the probe(s), a high salt concentration (e.g., greater than 1000 mM) and the absence of co-solvents.

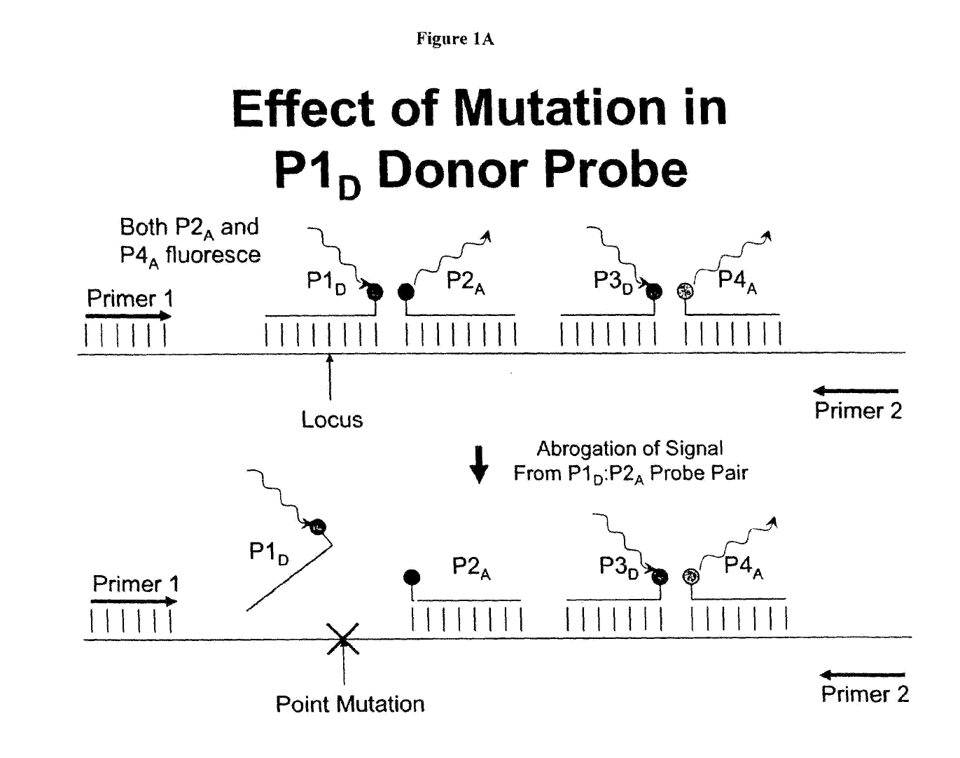

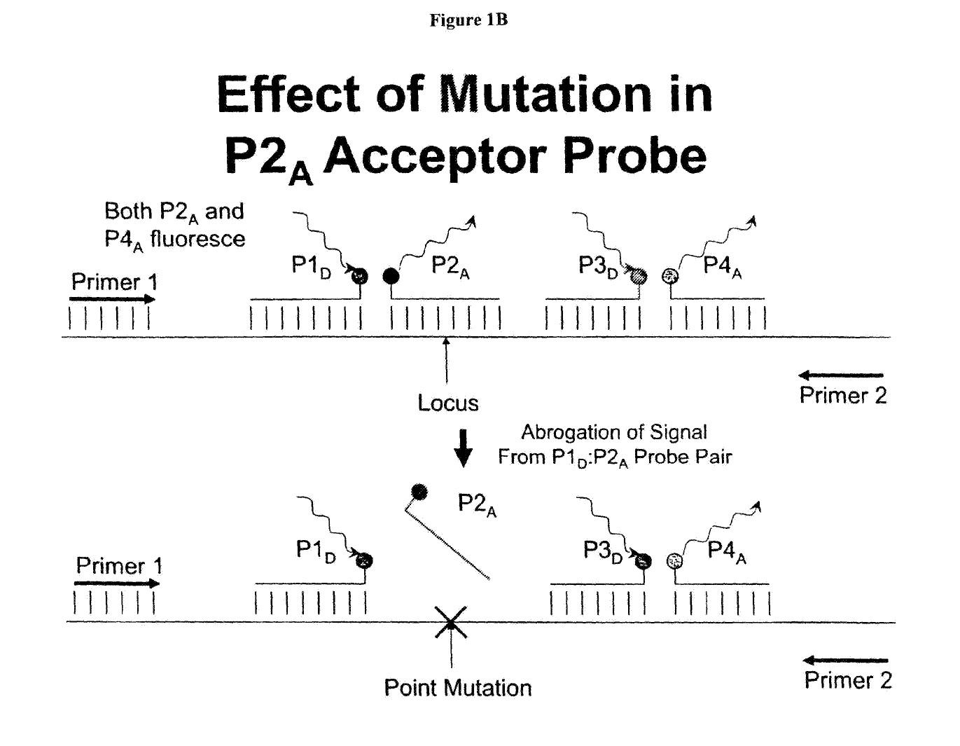

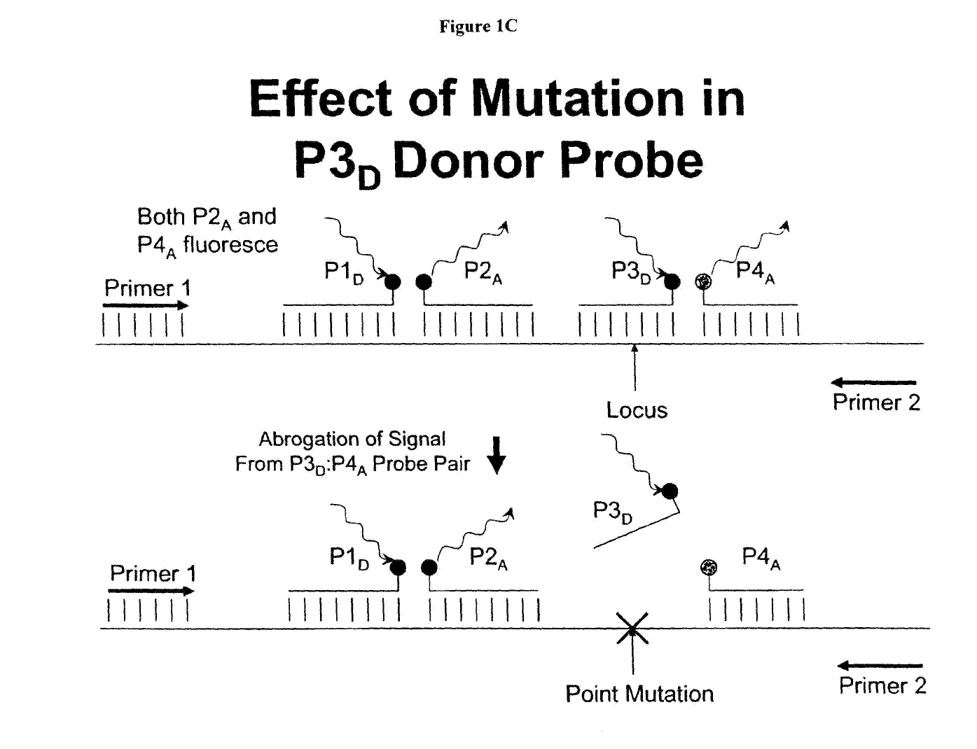

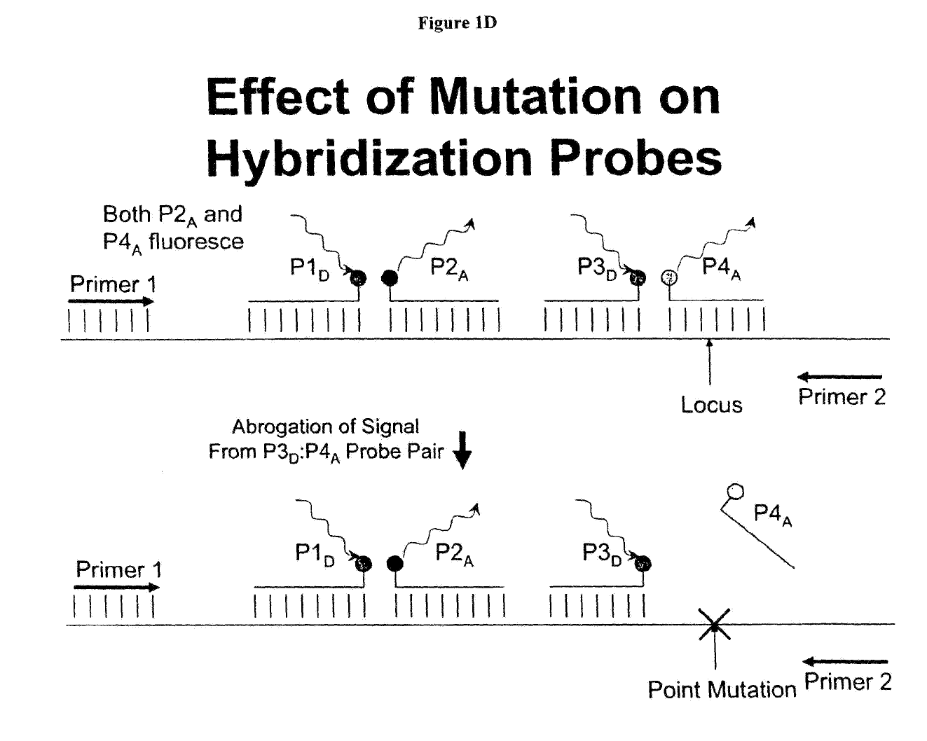

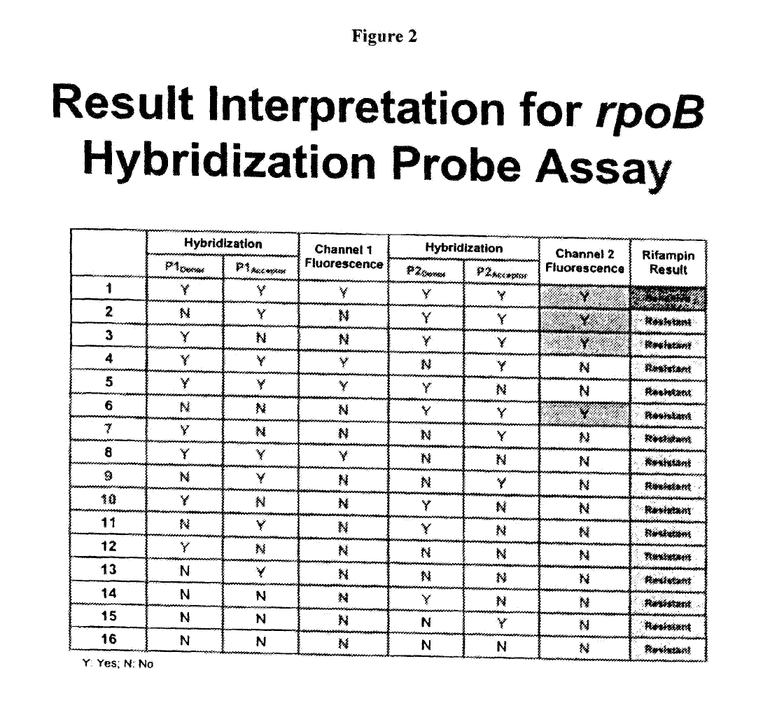

One, two, three, four, or more rpoB probes specific to the non-mutated Mycobacterium rpoB gene core region can be used in the methods described herein. In accordance with such methods, the rpoB probes specific to the non-mutated Mycobacterium rpoB gene core region hybridize to the rpoB gene core region such that the entirety of the core region is hybridized to rpoB probes when the rpoB gene core region does not comprise a mutation that confers resistance to the antimicrobial agent rifampin. For example, if a method described herein uses one rpoB probe specific to the non-mutated Mycobacterium rpoB gene core region, then the one rpoB probe will hybridize to the entire Mycobacterium rpoB gene core region so long as the Mycobacterium rpoB gene core region does not comprise a mutation; if the method uses two rpoB probes specific to the non-mutated Mycobacterium rpoB gene core region, then the two rpoB probes together will hybridize to the entire Mycobacterium rpoB gene core region so long as the Mycobacterium rpoB gene core region does not comprise a mutation; if the method uses three rpoB probes specific to the non-mutated Mycobacterium rpoB gene core region, then the three rpoB probes together will hybridize to the entire Mycobacterium rpoB gene core region so long as the Mycobacterium rpoB gene core region does not comprise a mutation; and if the method uses four rpoB probes specific to the non-mutated Mycobacterium rpoB gene core region, then the four rpoB probes together will hybridize to the entire Mycobacterium rpoB gene core region so long as the Mycobacterium rpoB gene core region does not comprise a mutation. FIG. 1 provides an exemplary diagram of how rpoB probes may hybridize to the Mycobacterium rpoB gene core region in the presence and absence of mutations within the Mycobacterium rpoB gene core region.

Mycobacterium Detector Probes

In certain embodiments, Mycobacterium detector probes are used. Mycobacterium detector probe comprise detectable labels, e.g., fluorescent labels, that allow for the determination of whether Mycobacterium is present in a sample. The Mycobacterium detector probes used in accordance with the methods described herein comprise a nucleic acid sequence that is complementary to a nucleic acid sequence of Mycobacterium, other than a nucleic acid sequence of the rpoB gene, e.g., IS6110, 16S rRNA, dnaJ or 85B antigen.

In a specific embodiment, a Mycobacterium detector probe is specific for a nucleic acid sequence of Mycobacterium that is conserved among all or multiple species or strains of Mycobacterium. In another embodiment, a Mycobacterium detector probe is specific for a nucleic acid sequence of a particular strain or species of Mycobacterium.

In some embodiments, a Mycobacterium detector probe is specific to a nucleic acid sequence of Mycobacterium that is abundantly available, e.g., 16S ribosomal RNA or 23S ribosomal RNA.

Probes Specific to Mycobacterium Genes Other than rpoB

In some embodiments, probes that are specific for genes of Mycobacterium other than the rpoB gene are used in accordance with the methods described herein. In accordance with such embodiments, the probes are specific for genes other than the rpoB gene, wherein said genes other than the rpoB gene can comprise mutations that confer resistance to one or more antimicrobial agents. Exemplary Mycobacterium genes for which probes may be designed include the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene. (see, e.g., Drobniewski and Wilson, 1998, J. Med. Microbiol., 47:189-196; Riska et al., 2000, Int. J. Tuberc. Lung. Dis., 4(2):S4-S10). In certain embodiments, the probes that are specific for genes of Mycobacterium other than the rpoB gene comprise a fluorescent moiety. In some embodiments, the probes that are specific for genes of Mycobacterium other than the rpoB gene comprise a fluorescent moiety and a quencher moiety positioned in the probe so that the quencher moiety and the fluorescent moiety are brought together in the absence of a complementary nucleic acid sequence. In some embodiments, the probes that are specific for genes of Mycobacterium other than the rpoB gene comprise a fluorescent donor moiety and a fluorescent acceptor moiety positioned in the probe so that the fluorescent donor moiety and the fluorescent acceptor moiety are brought together in the absence of a complementary nucleic acid sequence. In some embodiments, the probes that are specific for genes of Mycobacterium other than the rpoB gene form a hairpin loop in the absence of a complementary nucleic acid sequence.

In specific embodiments, the probes specific to genes of Mycobacterium other than the rpoB gene are designed so that they only hybridize to their complementary nucleic acid sequence when they are one hundred percent complementary, i.e., the probes will not bind to the nucleic acid sequence that they are complementary to if the nucleic acid sequence comprises a mutation.

Blocking Probes

In some embodiments, blocking probes are used in the methods described herein. Blocking probes may be designed to hybridize to (i) an rpoB probe that is being used in a method described herein; (ii) a nucleic acid sequence of the non-mutated rpoB gene core region; or (iii) a nucleic acid sequence outside of the rpoB gene core region.

In certain embodiments, a blocking probe is at least partially complementary to part of an rpoB probe, so that in the absence of a nucleic acid sample containing nucleic acid that hybridizes to the rpoB probe, the blocking probe hybridizes to the rpoB probe. In other embodiments, a blocking probe is at least partially complementary to a nucleic acid sequence of the non-mutated rpoB gene core region, wherein in the absence of one or more rpoB probes the blocking probe hybridizes to the non-mutated rpoB gene core region, and wherein in the presence of a non-mutated rpoB gene core region the rpoB probes hybridize to the non-mutated rpoB gene core region and not to the blocking probe. When a blocking probe is complementary to part of an rpoB probe, the blocking probe will hybridize to the rpoB probe only in the absence of the non-mutated region of the Mycobacterium rpoB gene core region to which that rpoB probe hybridizes. When a blocking probe is at least partially complementary to a nucleic acid sequence of the non-mutated rpoB gene core region, the blocking probe will hybridize to the nucleic acid sequence of the non-mutated rpoB gene core region only in the absence of the rpoB probe that hybridizes to the nucleic acid sequence of the non-mutated rpoB gene core region.

In some embodiments, a blocking probe is labeled with a quencher moiety that quenches a fluorescent moiety (e.g., a fluorescent donor moiety) of the rpoB probe to which the blocking probe binds.

Nucleic Acid Samples

The nucleic acid samples used in accordance with the methods described herein may be isolated from, obtained from, derived from, or taken from any source including, but not limited to, a subject (e.g., a human or animal).

In certain embodiments, a source is suspected of containing Mycobacterium, i.e., the source is suspected of being infected by and/or contaminated with Mycobacterium. In other embodiments, the source is suspected of containing Mycobacterium that is resistant to rifampin. In other embodiments, the source is suspected of containing multi-drug-resistant Mycobacterium.

In accordance with the methods described herein, the nucleic acid samples are lysed. In certain embodiments, the nucleic acid sample only contains Mycobacterium. In other embodiments, the nucleic acid sample contains host cells from a subject. In some embodiments, the nucleic acid sample contains host cells from a subject, wherein the Mycobacterium is localized within the host cells. In accordance with such embodiments, the host cells also are lysed. In some embodiments, one lysing agent is used to lyse a nucleic acid sample, e.g., one lysing agent can be used that lyses both Mycobacterium and host cells in the nucleic acid sample. In other embodiments, more than one lysing agents can be used to lyse a nucleic acid sample, e.g., one lysing agent can be used to lyse Mycobacterium in the nucleic acid sample and another lysing agent could be used to lyse host cells in the nucleic acid sample. In certain embodiments, a nucleic acid sample is lysed and the Mycobacterium nucleic acids in the nucleic acid sample are isolated and/or purified.

In certain embodiments, a nucleic acid sample is lysed prior to one or more of the methods described herein. In other embodiments, a nucleic acid sample is lysed as part of one or more of the methods described herein. Nucleic acid samples can be lysed by contacting them with a lysing agent. In some embodiments, lysing agents are biological agents, e.g., enzymes such as lysozyme, proteinase K or acheromopeptidase. In other embodiments, lysing agents are chemical agents such as chaotropes or detergents (e.g., guanidine isothiocyanate and sodium dodecyl sulphate). In further embodiments, lysing agents comprise one or more mechanical processes (e.g., pipettes, sonicators, French press). In yet further embodiments, lysing agents are combinations of biological and chemical agents and mechanical processes.

Nucleic acid samples used in accordance with one or more of the methods described herein may be processed/manipulated before their use in one or more of the methods described herein or as part of one or more of the methods described herein. For example, a nucleic acid sample may be stored (e.g., at room temperature, at 37° C., at 4° C., at −20° C., or at −70° C.), a nucleic acid sample may be washed (e.g., to remove undesired components), and/or a nucleic acid sample may be may be amplified. In certain embodiments, a nucleic acid sample is divided into portions, which can be processed/manipulated in the same manner or in a different manner. In some embodiments, a nucleic acid sample is divided into portions and at least one of the sample portions is used in accordance with one or more of the methods described herein, and the remaining nucleic acid sample portions are stored (e.g., at room temperature, at 37° C., at 4° C., at −20° C., or at −70° C.).

In certain embodiments, a nucleic acid sample is used in accordance with the methods described herein immediately after it is isolated or obtained from a source. In other embodiments, a nucleic acid sample is used in accordance with the methods described herein at most 30 minutes, 1 hour, 2 hours, 4 hours, 6 hours, 8 hours, 12 hours, or 24 hours after it is isolated or obtained from a source. In some embodiments, a portion of the nucleic acid sample is used in accordance with the methods described herein, and the remainder of the nucleic acid sample is retained for future use. In accordance with such embodiments, the remaining portion of the sample may be retained at room temperature, or stored at 4° C., −20° C., or −70° C.

In certain embodiments, a nucleic acid sample or portion thereof may be processed in a manner that condenses all or some of the non-liquid portion of the nucleic acid sample, e.g., by centrifugation. In some embodiments, nucleic acid samples of large size in terms of volume can be processed to condense the nucleic acid sample, after which some or all of the portion of the nucleic acid sample that does not comprise nucleic acid of Mycobacterium can be discarded and the retained nucleic acid sample can be used directly in one or more of the methods described herein or can be manipulated/processed prior to one or more of the methods described herein, e.g., the nucleic acid sample can be lysed, washed, resuspended in another medium, or amplified. In other embodiments, nucleic acid sample with non-liquid components other than Mycobacterium, e.g., blood components, may be processed to condense the non-liquid components other than Mycobacterium. The remainder of the nucleic acid sample then can be isolated and the non-liquid components other than Mycobacterium can be discarded.

Examples of subjects, e.g., humans, from which a nucleic acid sample may be obtained and utilized in accordance with the methods presented herein include, but are not limited to, asymptomatic subjects, subjects manifesting or exhibiting 1, 2, 3, 4 or more symptoms of a Mycobacterium infection, subjects clinically diagnosed as having a Mycobacterium infection, subjects predisposed to Mycobacterium infections (e.g., subjects that lead a lifestyle that predisposes them to Mycobacterium infections or increases the likelihood of contracting a Mycobacterium infection such as contacts of other subjects known to have tuberculosis), subjects suspected of having Mycobacterium infection, subjects undergoing therapy for a Mycobacterium infection, subjects with a Mycobacterium infection and at least one other condition (e.g., subjects with 2, 3, 4, 5 or more conditions), and subjects that have not been diagnosed with a Mycobacterium infection.

A nucleic acid sample can be obtained from any tissue or organ of a subject, or any secretion from a subject. Representative nucleic acid samples from a subject include, without limitation, bronchoalveolar lavage, a bronchial wash, a pharyngeal exudate, a tracheal aspirate, a blood sample, a serum sample, a plasma sample, a bone sample, a skin sample, a soft tissue sample, an intestinal tract specimen, a genital tract specimen, breast milk, a lymph sample, cerebrospinal fluid, pleural fluid, a sputum sample, a urine sample, a nasal secretion, tears, a bile sample, an ascites fluid sample, pus, stool, synovial fluid, vitreous fluid, a vaginal secretion, semen, and urethral samples. In some embodiments, two, three or more nucleic acid samples are obtained from a subject.

In one embodiment, the nucleic acid sample contains at least the minimum amount Mycobacterium nucleic acid required to perform one or more of the methods described herein. In other embodiments, the nucleic acid of Mycobacterium in the nucleic acid sample is amplified until it contains the minimum amount Mycobacterium nucleic acid required to perform one or more of the methods described herein.

In a specific embodiment, a nucleic acid sample comprises at least 1 colony forming unit (CFU), at least 10 CFU, at least 10² CFU, at least 10³ CFU, at least 10⁴ CFU, at least 10⁵ CFU, at least 10⁶ CFU, or at least 10⁷ CFU of Mycobacterium.

Nucleic Acid Amplification

Any method known in the art for amplifying nucleic acids can be used to amplify nucleic acids of Mycobacterium. For example, nucleic acids of Mycobacterium can be amplified using polymerase chain reaction (PCR) (see, e.g., Sambrook, et al., supra), strand displacement amplification (SDA), transcription-mediated amplification (TMA), nucleic acid sequence-based amplification (NASBA), rolling circle amplification, helix displacement amplification, or Loop-mediated isothermal amplification (LAMP).

In certain embodiments, the nucleic acids of Mycobacterium are amplified prior to performing one or more of the methods described herein. For example, the sample containing Mycobacterium is subjected to an amplification reaction, e.g., PCR, such that a nucleic acid sequence of one or more regions of the Mycobacterium is amplified prior to performing a method as described herein. In certain embodiments, the amplified nucleic acid sequence of Mycobacterium is the rpoB gene or a region of the rpoB gene, e.g., a region of the rpoB gene that comprises the rpoB gene core region. In other embodiments, the amplified nucleic acid sequence of Mycobacterium is a Mycobacterium gene other than the rpoB gene or a region of a Mycobacterium gene other than the rpoB gene, e.g., the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene.

In other embodiments, the nucleic acids of Mycobacterium are amplified as part of one or more of the methods described herein. For example, the sample containing Mycobacterium is subjected to an amplification reaction, e.g., PCR, such that a nucleic acid sequence of one or more regions of the Mycobacterium is amplified as part of a method described herein. In certain embodiments, the amplified nucleic acid sequence of Mycobacterium is the rpoB gene or a region of the rpoB gene, e.g., a region of the rpoB gene that comprises the rpoB gene core region. In other embodiments, the amplified nucleic acid sequence of Mycobacterium is a Mycobacterium gene other than the rpoB gene or a region of a Mycobacterium gene other than the rpoB gene, e.g., the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene.

5.3.1.1 Real-Time PCR

Real-time PCR, also called kinetic PCR, can enable both detection and quantification (as absolute number of copies or relative amount when normalized to DNA input or additional normalizing genes) of a specific sequence in a DNA sample.

In one method of real-time PCR, detection of amplified DNA can be performed using probes that fluoresce when hybridized with a complementary DNA. In accordance with this method, fluorescent probes are used to quantify only the DNA containing a nucleic acid sequence complementary to that of the probe; therefore, use of the probe significantly increases specificity, and allows quantification even in the presence of some non-specific DNA amplification. The use of sequence-specific probes allows for the identification of multiple regions of DNA in the same reaction by using specific probes with different-fluorescent labels. An increase in the nucleic acid sequence hybridized by a probe at each PCR cycle therefore causes a proportional increase in fluorescence.

Detection

In accordance with the methods described herein, detection of the fluorescence emission that results when a donor moiety transfers energy to an acceptor moiety or when fluorescence is emitted from a fluorescent moiety or other detectable label can be accomplished using any approach known in the art for measuring fluorescence emission, e.g., thermal cycler/fluorimeter (real-time PCR instrument), fluorescence laser scanner, or fluorescence microplate reader.

Real-Time PCR Instruments

So called “real-time” PCR involves the simultaneous amplification and detection of PCR products. Most commonly this is achieved using instruments that combine thermal cycling with fluorimetric detection. Excitation of fluorophores in the sample and detection of the resulting fluorescent emission spectra occur each cycle of the reaction, enabling a cumulative analysis of fluorescence intensity throughout the course of the reaction.

Microplate Readers

Microplate Readers are laboratory instruments designed to detect biological, chemical or physical events of samples in microtiter plates. Sample reactions can be assayed in, e.g., 6-1536 well format microtiter plates. A common detection mode for microplate assays includes fluorescence intensity detection.

In fluorescence intensity detection with microplate readers, a first optical system (excitation system) illuminates a sample using a specific wavelength. As a result of the illumination, the sample emits light (e.g., it fluoresces) and a second optical system (emission system) collects the emitted light, separates it from the excitation light (using a filter or monochromator system), and measures the signal using a light detector such as a photomultiplier tube (PMT).

Laser Scanning

Detectable labels used in the methods described herein can be detected using laser scanning, e.g., using a fluorescence laser scanner (see, e.g., in Schena et al., 1996, Genome Res. 6:639-645) or by laser scanning confocal microscopy (e.g., with a confocal laser-scanning microscope). Laser scanning approaches used in the methods described herein may be fluorescence-based, e.g., the laser excites the fluorescent moiety at a specific wavelength allowing for detection of the fluorescence emitted by the fluorescent moiety.

Diagnostic Methods

In certain embodiments, provided herein are methods for diagnosing a subject, e.g., a human, with a Mycobacterium infection comprising using one or more of the methods described herein. In specific embodiments, the methods presented herein can be used to diagnose a subject with an infection caused by Mycobacterium tuberculosis, Mycobacterium africanum, Mycobacterium bovis, Mycobacterium bovis BCG, or Mycobacterium microti. In certain embodiments, the methods presented herein can be used to diagnose a subject with tuberculosis. In other embodiments, the methods presented herein can be used to diagnose a subject with an infection of rifampin-resistant Mycobacterium. In other embodiments, the methods presented herein can be used to diagnose a subject with an infection of multi-drug-resistant Mycobacterium.

Also encompassed herein are methods of monitoring a Mycobacterium infection in a subject that has been diagnosed with a Mycobacterium infection. In accordance with such methods, a Mycobacterium infection in a subject can be monitored to assess whether the Mycobacterium acquires resistance to rifampin or becomes multi-drug-resistant Mycobacterium. The infection may be monitored by performing one or more of the methods described herein on a nucleic acid sample isolated or obtained from the subject. In certain embodiments, the infection is monitored at specific intervals (e.g., at least monthly) following the diagnosis of the infection until Mycobacterium is no longer detected. In certain embodiments, the infection is monitored after the patient has been administered one or more antimicrobial agents, e.g., rifampin or isoniazid, e.g., the infection is monitored weekly, bi-weekly, monthly, bi-monthly, every six months, or every year.

Selection of Therapy

The methods described herein allow for the determination of whether or not a Mycobacterium possesses a non-mutated Mycobacterium rpoB gene core region and is thus susceptible to the antimicrobial agent rifampin. The methods presented herein also allow for the determination of whether a Mycobacterium is resistant to other antimicrobial agents used in the treatment of Mycobacterium infections, e.g., other rifamycin derivatives (e.g., rifabutin; rifapentine) isoniazid, pyrazinamide, streptomycin, ethambutol, ethionamide, and ciprofloxacin. As such, the methods presented herein can provide information for the selection of an appropriate therapeutic regimen for a subject diagnosed with a Mycobacterium infection.

For example, detection of the presence of a non-mutated Mycobacterium rpoB gene core region in a sample may indicate that a subject should be treated with rifampin, whereas the failure to detect a non-mutated Mycobacterium rpoB gene core region in a sample may indicate that a subject should not be treated with rifampin. Additionally, determination of the susceptibility/resistance of a Mycobacterium in a sample from a subject to one or more antimicrobial agents can provide information for the treatment of other subjects, i.e., other subjects exhibiting similar symptoms of Mycobacterium infection may be administered the same one or more antimicrobial agents.

In addition to facilitating the appropriate therapeutic regimen for a subject, the determination of the susceptibility/resistance of a Mycobacterium to one or more antimicrobial agents may aid in reducing the transmission of the Mycobacterium from one subject to another.

Kits

Presented herein are kits comprising, in a container, one or more components required to determine the susceptibility/resistance of a Mycobacterium from a sample to one or more antimicrobial agents in accordance with one or more of the methods described herein.

In one embodiment, presented herein is a kit comprising a composition that comprises a set of probes, wherein at least five probes are rpoB probes that are specific for a non-mutated rpoB gene core region, and wherein (i) the first and third rpoB probes are labeled with a fluorescent donor moiety, (ii) the second and fourth rpoB probes are labeled with different fluorescent acceptor moieties, and (iii) the fifth probe is labeled with a fluorescent moiety; and wherein the rpoB probes hybridize to the non-mutated rpoB gene core region so that the fluorescent acceptor moieties can accept the transfer of energy from the fluorescent donor moieties.

In another embodiment, presented herein is a kit comprising a composition that comprises a set of probes, wherein at least five probes are rpoB probes that are specific for a non-mutated rpoB gene core region, and wherein (i) the first and third rpoB probes are labeled with a fluorescent donor moiety, (ii) the second and fourth rpoB probes are labeled with different fluorescent acceptor moieties, and (iii) the fifth probe is labeled with a fluorescent moiety and a quencher moiety, wherein, in the absence of the non-mutated rpoB gene core region to which the fifth rpoB probe is specific, the fifth rpoB probe forms a hairpin loop such that fluorescence from the fluorescent moiety is quenched; and wherein the rpoB probes hybridize to the non-mutated rpoB gene core region so that the fluorescent acceptor moieties can accept the transfer of energy from the fluorescent donor moieties.

In another embodiment, presented herein is a kit comprising a composition that comprises a set of probes, wherein at least four probes are rpoB probes that are specific for a non-mutated rpoB gene core region, and wherein the first and third rpoB probes are labeled with a fluorescent donor moiety and the second and fourth rpoB probes are labeled with different fluorescent acceptor moieties, and wherein the rpoB probes hybridize to the non-mutated rpoB gene core region so that the fluorescent acceptor moieties can accept the transfer of energy from the fluorescent donor moieties.

In another embodiment, presented herein is a kit comprising a composition that comprises a set of probes, wherein at least four probes are rpoB probes that are specific for a non-mutated rpoB gene core region, and wherein the first and second rpoB probes are labeled with different fluorescent moieties, the third rpoB probe is labeled with a fluorescent donor moiety, and the fourth rpoB probe is labeled with a fluorescent acceptor moiety, and wherein the third and fourth rpoB probes hybridize to the non-mutated rpoB gene core region so that the fluorescent acceptor moiety can accept the transfer of energy from the fluorescent donor moiety.

In another embodiment, presented herein is a kit comprising a composition that comprises a set of probes, wherein at least four probes are rpoB probes that are specific for a non-mutated rpoB gene core region, and wherein (i) the first and second rpoB probes comprise a fluorescent moiety and a quencher moiety, wherein, in the absence of the non-mutated rpoB gene core region to which the first and second rpoB probes are specific, the rpoB probes form a hairpin loop such that fluorescence from the fluorescent moieties is quenched, (ii) the third rpoB probe is labeled with a fluorescent donor moiety, and (iii) the fourth rpoB probe is labeled with a fluorescent acceptor moiety, and wherein the third and fourth rpoB probes hybridize to the non-mutated rpoB gene core region so that the fluorescent acceptor moiety can accept the transfer of energy from the fluorescent donor moiety.

In another embodiment, presented herein is a kit comprising a composition that comprises a set of probes, wherein at least four probes are rpoB probes specific for a non-mutated rpoB gene core region, and wherein the rpoB probes comprise different fluorescent moieties and a quencher moiety, and wherein, in the absence of the non-mutated rpoB gene core region to which the rpoB probes are specific, the rpoB probes form a hairpin loop such that fluorescence from the fluorescent moieties is quenched.

In another embodiment, presented herein is a kit comprising a composition that comprises a set of probes, wherein at least three probes are rpoB probes that are specific for a non-mutated rpoB gene core region, and wherein the first rpoB probe is labeled with a fluorescent moiety, the second rpoB probe is labeled with a fluorescent donor moiety, and the third rpoB probe is labeled with a fluorescent acceptor moiety, and wherein the second and third rpoB probes hybridize to the non-mutated rpoB gene core region so that the fluorescent acceptor moiety can accept the transfer of energy from the fluorescent donor moiety.

In another embodiment, presented herein is a kit comprising a composition that comprises a set of probes, wherein at least three probes are rpoB probes that are specific for a non-mutated rpoB gene core region, and wherein (i) the first rpoB probe comprises a fluorescent moiety and a quencher moiety, wherein, in the absence of the non-mutated rpoB gene core region to which the first rpoB probe is specific, the rpoB probe forms a hairpin loop such that fluorescence from the fluorescent moiety is quenched, (ii) the second rpoB probe is labeled with a fluorescent donor moiety, and (iii) the third rpoB probe is labeled with a fluorescent acceptor moiety, and wherein the second and third rpoB probes hybridize to the non-mutated rpoB gene core region so that the fluorescent acceptor moiety can accept the transfer of energy from the fluorescent donor moiety.

In another embodiment, presented herein is a kit comprising a composition that comprises a set of probes, wherein at least three probes are rpoB probes specific for a non-mutated rpoB gene core region, and wherein the rpoB probes comprise different fluorescent moieties and a quencher moiety, and wherein, in the absence of the non-mutated rpoB gene core region to which the rpoB probes are specific, the rpoB probes form a hairpin loop such that fluorescence from the fluorescent moieties is quenched.

In another embodiment, presented herein is a kit comprising a composition that comprises a set of probes, wherein at least two probes are rpoB probes specific for a non-mutated rpoB gene core region, and wherein the rpoB probes comprise different fluorescent moieties and a quencher moiety, and wherein, in the absence of the non-mutated rpoB gene core region to which the rpoB probes are specific, the rpoB probes form a hairpin loop such that fluorescence from the fluorescent moieties is quenched.

In another embodiment, presented herein is a kit comprising a composition that comprises a set of probes, wherein at least two probes are rpoB probes that are specific for a non-mutated rpoB gene core region, and wherein the first rpoB probe is labeled with a fluorescent donor moiety and the second rpoB probe is labeled with a fluorescent acceptor moiety, and wherein the rpoB probes hybridize to the non-mutated rpoB gene core region so that the fluorescent acceptor moiety accepts the transfer of energy from the fluorescent donor moiety.

In another embodiment, presented herein is a kit comprising a composition that comprises a set of probes, wherein at least one probe is an rpoB probe specific for a non-mutated rpoB gene core region, and wherein the rpoB probe comprises a fluorescent moiety and a quencher moiety, and wherein, in the absence of the non-mutated rpoB gene core region, the rpoB probe forms a hairpin loop such that fluorescence from the fluorescent moiety is quenched.

In another embodiment, presented herein is a kit comprising a composition that comprises a set of probes, wherein a first probe is an rpoB probe that is specific for a non-mutated rpoB gene core region and a second probe is complementary to a region of the rpoB gene outside the core region, and wherein the first probe is labeled with a fluorescent donor moiety and the second probe is labeled with a fluorescent acceptor moiety, and wherein the first and second probes hybridize to the rpoB gene so that the fluorescent acceptor moiety can accept the transfer of energy from the fluorescent donor moiety.

In another embodiment, presented herein is a kit comprising a composition that comprises a set of probes, wherein a first probe is complementary to a region of the rpoB gene outside of the core region and second and third probes are rpoB probes that are specific for a non-mutated rpoB gene core region, and wherein the first probe is labeled with a fluorescent donor moiety, the second probe is labeled with a fluorescent acceptor moiety, and the third probe is labeled with a fluorescent moiety, and wherein the first and second probes hybridize to rpoB gene so that the fluorescent acceptor moiety can accept the transfer of energy from the fluorescent donor moiety.

In another embodiment, presented herein is a kit comprising a composition that comprises a set of probes, wherein a first probe is complementary to a region of the rpoB gene outside of the core region and second, third, and fourth probes are rpoB probes that are specific for a non-mutated rpoB gene core region, and wherein the first and third probes are labeled with a fluorescent donor moiety and the second and fourth probes are labeled with different fluorescent acceptor moieties, and wherein the probes hybridize to the rpoB gene so that the fluorescent acceptor moieties can accept the transfer of energy from the fluorescent donor moieties.

In another embodiment, presented herein is a kit comprising a composition that comprises a set of probes, wherein a first probe is complementary to a region of the rpoB gene outside of the core region and second, third, fourth, and fifth probes are rpoB probes that are specific for a non-mutated rpoB gene core region, and wherein the first and third probes are labeled with a fluorescent donor moiety, the second and fourth probes are labeled with different fluorescent acceptor moieties, and the fifth probe is labeled with a fluorescent moiety, and wherein the probes hybridize to the rpoB gene so that the fluorescent acceptor moieties can accept the transfer of energy from the fluorescent donor moieties.

In some embodiments, the kits provided herein comprise a Mycobacterium detector probe.

In certain embodiments, the kits provided herein comprise one or more reagents for use in lysing a nucleic acid sample; amplifying a nucleic acid sample (e.g., PCR primers); isolating a nucleic acid sample; purifying a nucleic acid sample; and/or concentrating a nucleic acid sample.

In a specific embodiment, the kits provided herein comprise one or more sets of primers for use in amplifying the Mycobacterium rpoB gene or a region thereof (e.g., the Mycobacterium rpoB gene core region). In another specific embodiment, the kits provided herein comprise one or more sets of primers for use in amplifying a Mycobacterium gene or a region thereof other than the Mycobacterium rpoB gene, e.g., one or more sets of primers for amplifying the inhA gene, the katG gene, the ahpC gene, the mabA gene, the oxyR gene, the pncA gene, the rrs gene, the rpsL gene, the embA gene, the embB gene, the embC gene, the gyrA gene, the gyrB gene, or the nor gene of Mycobacterium, or a region thereof. In another specific embodiment, the kits provided herein comprise one or more buffers, e.g., buffers for use in washing during hybridization.

In other embodiments, the kits provided herein comprise one or more blocking probes, wherein the one or more blocking probes (i) are at least partially complementary to an rpoB probe that hybridizes to a non-mutated Mycobacterium rpoB gene core region, so that in the absence of a nucleic acid sample containing nucleic acid that hybridizes to the rpoB probe, the blocking probe hybridizes to the rpoB probe; or (ii) are at least partially complementary to a nucleic acid sequence of the Mycobacterium rpoB gene core region, wherein in the presence of a non-mutated Mycobacterium rpoB core region, the rpoB probe hybridizes to the core region and not to the blocking probe. In some embodiments, the blocking probes comprise a self-complementary region such that, in the absence of a nucleic acid sample containing nucleic acid that hybridizes to the blocking probe, the blocking probe forms a hairpin loop. In some embodiments, the blocking probes are labeled with a quencher moiety that quenches a fluorescent moiety (e.g., a fluorescent donor moiety) of the rpoB probe to which the blocking probe binds.

The kits presented herein may comprise instructions for using the kits to detect a non-mutated Mycobacterium rpoB gene core region. In a specific embodiment, the instructions recommend that positive and negative controls are run in parallel with test samples. In some embodiments, the kits presented herein comprise a sample that is known not to hybridize with one or more of the probes provided in the kit (i.e., a negative control). In other embodiments, the kits presented herein comprise a sample that is known to hybridize with one or more of the probes provided in the kit (i.e., a positive control). In yet other embodiments, the kits presented herein comprise a sample that is known to hybridize with one or more of the probes provided in the kit (i.e., a positive control) and a sample that is known not to hybridize with one or more of the probes provided in the kit (i.e., a negative control).

Systems

Presented herein are systems, e.g., automated systems, comprising a kit or a component(s) of the kits presented herein and a computer program product for use in conjunction with a computer system. In such systems, the computer program product can comprise a computer readable storage medium and a computer program mechanism embedded therein. The computer program mechanism may comprise instructions for evaluating whether a non-mutated Mycobacterium rpoB gene core region is present, whether Mycobacterium is present, and/or whether a mutation exists in a Mycobacterium gene other than rpoB.

Some systems presented herein comprise a kit or one or more components of the kits presented herein, a computer having a central processing unit and a memory coupled to the central processing unit. Some systems presented herein comprise a kit or one or more components of the kits presented herein, a computer readable medium, a computer having a central processing unit, and a memory coupled to the central processing unit. The memory stores instructions for evaluating whether a non-mutated Mycobacterium rpoB gene core region is present, whether Mycobacterium is present, and/or whether a mutation exists in a Mycobacterium gene other than rpoB. In some embodiments, the memory comprises instructions for transmitting the results of a method presented herein to a remote computer and the remote computer includes instructions for evaluating whether a non-mutated Mycobacterium rpoB gene core region is present, whether Mycobacterium is present, and/or whether a mutation exists in a Mycobacterium gene other than rpoB.

In some embodiments, presented herein is a computer system comprising a computer readable medium comprising the results of evaluating whether a non-mutated Mycobacterium rpoB gene core region is present, whether Mycobacterium is present, and/or whether a mutation exists in a Mycobacterium gene other than rpoB. In some embodiments, a computer system presented herein comprises:
a central processing unit;
a main non-volatile storage unit, for example, a hard disk drive, for storing software and data, the storage unit controlled by storage controller;
a system memory, such as high speed random-access memory (RAM), for storing system control programs, data and application programs, comprising programs and data loaded from non-volatile storage unit, and may also include a read-only memory (ROM);
a user interface, comprising one or more input devices (e.g., a keyboard) and display or other output device;
a network interface card for connecting to any wired or wireless communication network (e.g., a wide area network such as the Internet);
an internal bus for interconnecting the aforementioned elements of the system; and
a power source to power the aforementioned elements.

Operation of the computer can be controlled primarily by an operating system, which is executed by a central processing unit. The operating system can be stored in the system memory. In addition to the operating system, an implementation system may include: a file system for controlling access to the various files and data structures presented herein; a training data set for use in the construction of one or more decision rules in accordance with the methods presented herein; a data analysis algothrithm module for processing training data and constructing decision rules; one or more decision rules; a profile evaluation module for determining whether a non-mutated Mycobacterium rpoB gene core region is present, whether Mycobacterium is present, and/or whether a mutation exists in a Mycobacterium gene other than rpoB.

The computer may comprise software program modules and data structures. Each of the data structures can comprise any form of a data storage system, including, but not limited to, a flat ASCII or binary file, an Excel spreadsheet, a relational database (e.g., SQL), or an on-line analytical processing (OLAP) database (e.g., MDX and/or variants thereof). In some embodiments, such data structures are each in the form of one or more databases that include a hierarchical structure (e.g., a star schema). In some embodiments, such data structures are each in the form of databases that do not have explicit hierarchy (e.g., dimension tables that are not hierarchically arranged).

In some embodiments, each of the data structures stored or accessible to the computer system are single data structures. In other embodiments, such data structures in fact comprise a plurality of data structures (e.g., databases, files, archives) that may or may not all be hosted by the same computer. For example, in some embodiments, a training data set may comprise a plurality of Excel spreadsheets that are stored either on the computer and/or computers that are addressable by the computer across a wide area network. In another example, a training set may comprise a database that is either stored on the computer or is distributed across one or more computers that are addressable by the computer across a wide area network.

It will be appreciated that many of the modules and data structures mentioned above can be located on one or more remote computers. For example, in some embodiments, web service-type implementations are used. In such embodiments, an evaluation module can reside on a client computer that is in communication with the computer via a network. In some embodiments, a profile evaluation module can be an interactive web page.

In some embodiments, a training data set and/or decision rules are on a single computer and in other embodiments, one or more of such data structures and modules are hosted by one or more remote computers. Any arrangement of the data structures and software modules on one or more computers is within the scope the systems presented herein so long as these data structures and software modules are addressable with respect to each other across a network or by other electronic means.

In some embodiments, a digital signal embodied on a carrier wave comprises data with respect to a method presented herein. In some embodiments, a digital signal embodied on a carrier wave comprises a determination as to whether a non-mutated Mycobacterium rpoB gene core region is present, whether Mycobacterium is present, and/or whether a mutation exists in a Mycobacterium gene other than rpoB. In some embodiments, a graphical user interface is provided for determining whether a non-mutated Mycobacterium rpoB gene core region is present, whether Mycobacterium is present, and/or whether a mutation exists in a Mycobacterium gene other than rpoB. The graphical user interface may comprise a display field for displaying a result encoded in a digital signal embodied on a carrier wave received from a remote computer.

### EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. A method for determining the presence of a non-mutated Mycobacterium rpoB gene core region, comprising:
(a) contacting a nucleic acid sample with a composition comprising a set of probes, wherein at least four probes are rpoB probes that are specific for a non-mutated rpoB gene core region, under conditions that allow hybridization of the rpoB probes to the core region, and wherein the first and third rpoB probes are labeled with a fluorescent donor moiety and the second and fourth rpoB probes are labeled with different fluorescent acceptor moieties, and wherein the rpoB probes hybridize to the non-mutated rpoB gene core region so that the fluorescent acceptor moieties can accept the transfer of energy from the fluorescent donor moieties; and
(b) assaying for the presence of fluorescence emission that results when both donor moieties transfer energy to both acceptor moieties, wherein the presence of the fluorescence emission that results when both donor moieties transfer energy to both acceptor moieties indicates the presence of a non-mutated rpoB gene core region, and wherein the absence of or reduction in the fluorescence emission that results when both donor moieties transfer energy to both acceptor moieties indicates the absence of a non-mutated rpoB gene core region.

2. (canceled)

3. The method of claim 1, wherein the Mycobacterium is Mycobacterium tuberculosis, Mycobacterium africanum, Mycobacterium bovis, Mycobacterium bovis BCG, or Mycobacterium microti.

4. The method of claim 1, wherein the presence of a non-mutated rpoB gene core region indicates that the Mycobacterium is susceptible to rifampin, and wherein the absence of a non-mutated rpoB gene core region indicates that the Mycobacterium is resistant to rifampin.

5. The method of claim 1, wherein the set of probes further comprises a blocking probe that is at least partially complementary to an rpoB probe, so that in the absence of a nucleic acid sample containing nucleic acid that hybridizes to the rpoB probe, the blocking probe hybridizes to the rpoB probe.

6. The method of claim 5, wherein the blocking probe comprises a quencher moiety, wherein the quencher moiety quenches the fluorescence emitted by the fluorescent donor moiety or fluorescent acceptor moiety of the rpoB probe.

7. The method of claim 1, wherein the set of probes further comprises a blocking probe that is at least partially complementary to a nucleic acid sequence of the non-mutated rpoB gene core region, wherein in the absence of the rpoB probes, the blocking probe hybridizes to the non-mutated rpoB gene core region, and wherein in the presence of a non-mutated rpoB gene core region, the non-mutated rpoB gene core region hybridizes to the rpoB probes and not to the blocking probe.

8. The method of claim 1, wherein at least one rpoB probe comprises a self-complementary region that in the absence of a nucleic acid sample containing nucleic acid that hybridizes to the rpoB probe, the rpoB probe forms a hairpin loop.

9. The method of claim 1, wherein the core region comprises 81 nucleotides.

10. (canceled)

11. The method of claim 1, wherein the nucleic acid sample is isolated or obtained from a biological fluid or tissue from a subject.

12. The method of claim 11, wherein the subject is a human.

13. The method of claim 12, wherein the biological fluid or tissue is a bronchoalveolar lavage, a bronchial wash, a pharyngeal exudate, a tracheal aspirate, a blood sample, a serum sample, a plasma sample, a bone sample, a skin sample, a soft tissue sample, an intestinal tract specimen, a stool sample, a genital tract specimen, breast milk, a lymph sample cerebrospinal fluid, pleural fluid, a sputum sample, a urine sample, a nasal secretion, tears, a bile sample, an ascites fluid sample, pus, synovial fluid, vitreous fluid, a vaginal secretion, semen, or a urethral sample.

14. The method of claim 1, wherein the nucleic acid sample comprises amplified nucleic acids.

15. The method of claim 14, wherein the amplified nucleic acids were generated using PCR, SDA, TMA, NASBA, rolling circle amplification, helix displacement amplification, or LAMP.

16. (canceled)

17. (canceled)

18. The method of claim 1, wherein the nucleic acid sample further comprises a lysing agent.

19. The method of claim 18, wherein the lysing agent is lysozyme, heat, sonication, pressure, or a chaotropic agent.

20. The method of claim 1, wherein the fluorescent acceptor moieties or the fluorescent donor moieties are selected from the following: FITC, ROX, GFP, Cy5, Cy5.5, Cy3, Cy3B, GFP, YFP, RFP, CFP, Rhodamine Red, Texas Red, Bodipy, IDR700, LightCycler 610, LightCycler 640, LightCycler 670, LightCycler 705, and TAMRA.

21. (canceled)

22. (canceled)

23. (canceled)

24. A kit comprising a composition that comprises a set of probes, wherein at least four probes are rpoB probes that are specific for a non-mutated rpoB gene core region of Mycobacterium, and wherein the first and third rpoB probes are labeled with a fluorescent donor moiety and the second and fourth rpoB probes are labeled with two different fluorescent acceptor moieties, and wherein the rpoB probes hybridize to the non-mutated rpoB gene core region so that the fluorescent acceptor moieties can accept the transfer of energy from the fluorescent donor moieties.

25. The kit of claim 24, wherein the Mycobacterium is Mycobacterium tuberculosis, Mycobacterium africanum, Mycobacterium bovis, Mycobacterium bovis BCG, or Mycobacterium microti.

26. The kit of claim 24, wherein at least one rpoB probe comprises a self-complementary region that in the absence of a nucleic acid sample containing nucleic acid that hybridizes to the rpoB probe, the rpoB probe forms a hairpin loop.

27. The kit of claim 26, wherein the at least one rpoB probe comprises a quencher moiety.

28. The kit of claim 24, wherein the set of probes further comprises one or more blocking probes.

29. The kit of claim 28, wherein the one or more blocking probes
(i) are at least partially complementary to an rpoB probe, so that in the absence of a nucleic acid sample containing nucleic acid that hybridizes to the rpoB probe, the blocking probe hybridizes to the rpoB probe; and/or
(ii) are at least partially complementary to a nucleic acid sequence of the core region, wherein in the absence of the rpoB probes, the blocking probe hybridizes to the non-mutated rpoB gene core region, and wherein in the presence of a non-mutated rpoB gene core region of Mycobacterium, the non-mutated rpoB gene core region hybridizes to the rpoB probes and not to the blocking probe.

30. The kit of claim 29, wherein the blocking probe that is at least partially complementary to an rpoB probe comprises a quencher moiety, wherein the quencher moiety quenches the fluorescence emitted by the fluorescent donor moiety and/or fluorescent acceptor moiety of the rpoB probe.

31. (canceled)

32. (canceled)

33. (canceled)

34. (canceled)

35. (canceled)

36. (canceled)
